(19) United States
(12) Patent Application Publication
(10) Pub. No.: US 2024/0239864 A1
(43) Pub. Date: Jul. 18, 2024

(54) HLA CLASS I-RESTRICTED T CELL RECEPTORS AGAINST CD22

(71) Applicant: The United States of America,as represented by the Secretary,Department of Health and Human Services, Bethesda, MD (US)

(72) Inventors: Kazusa Ishii, Bethesda, MD (US); Christian Hinrichs, Bethesda, MD (US)

(73) Assignee: The United States of America,as represented by the Secretary,Department of Health and Human Services, Bethesda, MD (US)

(21) Appl. No.: 18/277,521

(22) PCT Filed: Feb. 16, 2022
(86) PCT No.: PCT/US2022/016561

§ 371 Date: Aug. 16, 2023

Related U.S. Application Data
(60) Provisional application No. 63/149,795, filed on Feb. 16, 2021.

(51) Int. Cl. C07K 14/725; A61K 39/00; A61P 35/00; C12N 5/0783; G01N 33/574

(52) U.S. Cl. CPC C07K 14/7051; A61K 39/4611; A61K 39/4632; A61K 39/464413; A61P 35/00; C12N 5/0636; G01N 33/57484; C12N 2510/00; G01N 2333/7051

(57) ABSTRACT

Disclosed are isolated or purified T cell receptors (TCRs), wherein the TCRs have antigenic specificity for a CD22 amino acid sequence presented by a human leukocyte antigen (HLA) Class I molecule. Related polypeptides and proteins, as well as related nucleic acids, recombinant expression vectors, host cells, populations of cells, and pharmaceutical compositions are also provided. Also disclosed are methods of detecting the presence of cancer in a mammal and methods of treating or preventing cancer in a mammal.

### CROSS-REFERENCE TO RELATED APPLICATION

This patent application claims the benefit of co-pending U.S. Provisional Patent Application No. 63/149,795 filed Feb. 16, 2021, which is incorporated by reference herein in its entirety.

### STATEMENT REGARDING FEDERALLY SPONSORED RESEARCH OR DEVELOPMENT

This invention was made with Government support under project number 1 ZIA BC011479 by the National Institutes of Health, National Cancer Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 67,778 Byte ASCII (Text) file named “759322_ST25” dated Feb. 16, 2022.

### BACKGROUND OF THE INVENTION

Some cancers may have very limited treatment options, particularly when the cancer becomes metastatic and unresectable. Despite advances in treatments such as, for example, surgery, chemotherapy, and radiation therapy, the prognosis for many cancers, such as, for example, lymphomas, leukemias, and epithelial cancers, including Hodgkin lymphoma, T/NK cell lymphoma, post-transplant lymphoproliferative disorders, nasopharyngeal cancer, and gastric cancer, may be poor. Accordingly, there exists an unmet need for additional treatments for cancer.

### BRIEF SUMMARY OF THE INVENTION

An aspect of the invention provides an isolated or purified T-cell receptor (TCR) comprising the amino acid sequences of (a) SEQ ID NOs: 1-3, (b) SEQ ID NOs: 4-6, or (c) SEQ ID NOs: 1-6, wherein the TCR has antigenic specificity for a CD22 amino acid sequence presented by a human leukocyte antigen (HLA) Class I molecule.

Another aspect of the invention provides an isolated or purified polypeptide comprising a functional portion of the inventive TCR, wherein the functional portion comprises the amino acid sequences of (a) SEQ ID NOs: 1-3, (b) SEQ ID NOs: 4-6, or (c) SEQ ID NOs: 1-6.

Still another aspect of the invention provides an isolated or purified protein comprising at least one of the inventive polypeptides.

Further aspects of the invention provide nucleic acids, recombinant expression vectors, host cells, populations of cells, and pharmaceutical compositions relating to the inventive TCRs, polypeptides, and proteins.

An aspect of the invention provides an isolated or purified nucleic acid comprising, from 5′ to 3′, a first nucleic acid sequence and a second nucleotide sequence, wherein the first and second nucleotide sequence, respectively, encode the amino sequences of SEQ ID NOs: 7, and 8 or 9; 8 or 9, and 7; 10 and 11; or 11 and 10.

Methods of detecting the presence of cancer in a mammal, methods of treating or preventing cancer in a mammal, methods of inducing an immune response against a cancer in a mammal, methods of producing a host cell expressing a TCR that has antigenic specificity for a CD22 amino acid sequence, e.g., the peptide of SEQ ID NO: 12, and methods of producing the inventive TCRs, polypeptides, and proteins, are further provided by aspects of the invention.

Additional aspects are as described herein.

### BRIEF DESCRIPTION OF THE DRAWINGS

Results shown in the figures as described below are in accordance with aspects of the invention.

FIG. 1 presents a graph showing surface HLA-A2 expression of T2 cells loaded with the indicated peptides or without exogenous peptides (T2 only), assessed using flow cytometry analysis.

FIG. 2 presents a flow diagram of the allogeneic TCR repertoire screening method as described in Example 1.

FIG. 3 presents flow cytometry plots showing enrichment of HLA-A*02:01 CD22 p228-236 tetramer-binding CD8+ T cells after repeated stimulation with A2+ DC/K562A2 loaded with CD22 p228-236.

FIG. 4A presents flow cytometry plots of polyclonal T cells raised with repeated stimulation with A2+DC/K562A2, that were co-cultured with K562 for 4 hours: The T cells were co-cultured with K562 for 4 hours, then intracellular IFNγ production along with surface p-MHC tetramer binding was assessed using flow cytometry analysis. The top panel is an example of a cross-reactive TCR that demonstrated non-specific HLA-A2 recognition, and the bottom panel is the D146W8 TCR.

FIG. 4B presents flow cytometry plots of polyclonal T cells raised with repeated stimulation with A2+DC/K562A2, that were then co-cultured with K562A2 for 4 hours: The T cells were co-cultured with K562A2 for 4 hours, then intracellular IFNγ production along with surface p-MHC tetramer binding was assessed using flow cytometry analysis. The top panel is an example of a cross-reactive TCR that demonstrated non-specific HLA-A2 recognition, and the bottom panel is the D146W8 TCR.

FIG. 4C presents flow cytometry plots of polyclonal T cells raised with repeated stimulation with A2+DC/K562A2, that were then co-cultured with K562A2 loaded with cognate peptides CD22 p228-236. K562A2 were loaded with 1 μM of peptide CD22 p228-236 for 30 minutes, then the T cells were co-cultured with the peptide-loaded K562A2 for 4 hours, then intracellular IFNγ production along with surface p-MHC tetramer binding was assessed using flow cytometry analysis. The top panel is an example of a cross-reactive TCR that demonstrated non-specific HLA-A2 recognition, and the bottom panel is the D146W8 TCR.

FIG. 4D presents flow cytometry plots of polyclonal T cells raised with repeated stimulation with A2+DC/K562A2, that were then co-cultured with K562A2 loaded with irrelevant peptides HPV-16 E7p11-19: K562A2 were loaded with 1 μM of an irrelevant peptide HPV-16 E7 p11-19 for 30 minutes, then the T cells were co-cultured with the peptide-loaded K562A2 for 4 hours, then intracellular IFNγ production along with surface p-MHC tetramer binding was assessed using flow cytometry analysis. The top panel is an example of a cross-reactive TCR that demonstrated non-specific HLA-A2 recognition, and the bottom panel is the D146W8 TCR.

FIG. 4E presents flow cytometry plots of polyclonal T cells raised with repeated stimulation with A2+DC/K562A2 that were then co-cultured with K562A2 expressing human CD22 protein: K562A2 were retrovirally transduced to express full-length human CD22 protein (called K562A2-FL.CD22 in the figure). T cells were co-cultured with the K562A2-FL.CD22 for 4 hours, then intracellular IFNγ production along with surface p-MHC tetramer binding was assessed using flow cytometry analysis. The top panel is an example of a cross-reactive TCR that demonstrated non-specific HLA-A2 recognition, and the bottom panel is the D146W8 TCR.

FIG. 4F presents flow cytometry plots of polyclonal T cells raised with repeated stimulation with A2+DC/K562A2 that were then co-cultured with K562A2 expressing irrelevant human protein, CD19: K562A2 were retrovirally transduced to express full-length human CD19 protein (called K562A2-FL.CD19 in the figure). T cells were co-cultured with the K562A2-FL.CD19 for 4 hours, then intracellular IFNγ production along with surface p-MHC tetramer binding was assessed using flow cytometry analysis. The top panel is an example of a cross-reactive TCR that demonstrated non-specific HLA-A2 recognition, and the bottom panel is the D146W8 TCR.

FIG. 4G presents flow cytometry plots of polyclonal T cells raised with repeated stimulation with A2+DC/K562A2. T cells were cultured for 4 hours without target cells. The top panel is an example of a cross-reactive TCR that demonstrated non-specific HLA-A2 recognition, and the bottom panel is the D146W8 TCR.

FIG. 4H presents flow cytometry plots of polyclonal T cells raised with repeated stimulation with A2+DC/K562A2, then stimulated with PMA/ionomycin for 4 hours. The top panel is an example of a cross-reactive TCR that demonstrated non-specific HLA-A2 recognition, and the bottom panel is the D146W8 TCR.

FIG. 5 presents a schematic of the TCR expression cassette constructed in Example 2.

FIG. 6A presents a flow cytometry plot showing human polyclonal T-lymphocytes retrovirally transduced to express anti-CD22 TCR, D146W8. Transduced T cells were stained with CD22 p228-236 p-MHC tetramer, and antibodies against CD8, CD4, and mTRBC.

FIG. 6B presents a flow cytometry plot showing human polyclonal T-lymphocytes retrovirally transduced to express anti-E7 TCR T cells. Transduced T cells were stained with CD22 p228-236 p-MHC tetramer, and antibodies against CD8, CD4, and mTRBC.

FIG. 6C presents a flow cytometry plot showing untransduced T cells stained with stained with CD22 p228-236 p-MHC tetramer, and antibodies against CD8, CD4, and mTRBC.

FIG. 6D presents a flow cytometry plot showing human polyclonal T-lymphocytes retrovirally transduced to express anti-CD22 TCR, D146W8. Transduced T cells were stained with HPV-16 E7p11-19 p-MHC tetramer, and antibodies against CD8, CD4, and mTRBC.

FIG. 6E presents a flow cytometry plot showing human polyclonal T-lymphocytes retrovirally transduced to express anti-E7 TCR. Transduced T cells were stained with HPV-16 E7p11-19 p-MHC tetramer, and antibodies against CD8, CD4, and mTRBC.

FIG. 6F presents a flow cytometry plot showing untransduced T cells stained with HPV-16 E7p11-19 p-MHC tetramer, and antibodies against CD8, CD4, and mTRBC.

FIG. 7 is a graph of IFNγ levels produced by T cells transduced with an anti-CD22 TCR or anti-E7 TCR that were stimulated with K562A2 loaded with varying concentrations of CD22 p228-236 or E7 p11-19.

FIG. 8A is a bar graph showing IFNγ levels produced by T cells transduced with an anti-CD22 TCR or anti-E7 TCR, co-cultured with the indicated K562-based target cells.

FIG. 8B is a bar graph showing IFNγ levels produced by T cells transduced with an anti-CD22 TCR or anti-E7 TCR, co-cultured with the indicated EBV-LCL target cells.

FIG. 8C is a bar graph showing IFNγ levels produced by T cells transduced with an anti-CD22 TCR or anti-E7 TCR, co-cultured with the indicated target cells.

FIGS. 9A, 9B, and 9C are bar graphs showing the percent of CD8+ cells being IFNγ+/TNFα−, IFNγ+/TNFα+, IFNγ−/TNFα+, respectively, after T cells transduced with an anti-CD22 TCR or anti-E7 TCR were co-cultured with indicated cell lines on the x-axis.

FIG. 10 is a bar graph showing IFNγ levels produced by T cells transduced with an anti-CD22 TCR or an anti-E7 TCR co-cultured with K562A2 loaded with or without 1 μM of peptide as shown.

FIGS. 11A and 11B are bar graphs showing IFNγ levels of T cells transduced with anti-CD22 TCR co-cultured with K562A2 loaded with the 1 μM of peptides as indicated. FIG. 11A presents SEQ ID NOS: 44-107 from left to right; FIG. 11B presents SEQ ID NOS: 108-170 from left to right.

FIG. 12 is a bar graph showing that the anti-CD22 TCR recognized the intended epitope with higher sensitivity compared to irrelevant peptides identified in FIGS. 11A and 11B.

FIGS. 13 and 14 present images (FIG. 13) and a line graph (FIG. 14) showing that the anti-CD22 TCR-transduced T cells mediate in vivo cytotoxicity as demonstrated by cell-dose-dependent reduction in the leukemia burden. For FIG. 14, n.s. is not significant, * is p=0.02, ** is p<0.001.

### DETAILED DESCRIPTION OF THE INVENTION

An aspect of the invention provides an isolated or purified TCR having antigenic specificity for a CD22 amino acid sequence, e.g., CD22 (p228-236) amino acid sequence FLSNDTVQL (SEQ ID NO: 12), presented by a human leukocyte antigen (HLA) Class I molecule. A wide range of B-lymphoid malignancies, such as non-Hodgkin lymphomas (NHL) and acute lymphoblastic leukemia, express CD22. Hereinafter, references to a “TCR” also refer to functional portions and functional variants of the TCR, unless specified otherwise.

In aspects of the invention, the inventive TCRs are able to recognize CD22 presented by an HLA Class I molecule. In this regard, the TCR may elicit an immune response upon binding to CD22-derived peptides presented in the context of an HLA Class I molecule.

In an aspect of the invention, the HLA Class I molecule is an HLA-A molecule. The HLA-A molecule is a heterodimer of an α chain and 02 microglobulin. The HLA-A α chain may be encoded by an HLA-A gene. 02 microglobulin binds non-covalently to the alpha chain to build the HLA-A complex. The HLA-A molecule may be any HLA-A molecule. In aspects of the invention, the HLA Class I molecule is an HLA-A02 molecule. The HLA-A02 molecule may be any HLA-A02 molecule. Examples of HLA-A02 molecules may include, but are not limited to, those expressed by the HLA-A*02:01, HLA-A*02:02, HLA-A*02:03, HLA-A*02:05, HLA-A*02:06, HLA-A*02:07, and HLA-A*02:11 alleles. Preferably, the HLA-A02 molecule is expressed by the HLA-A*02:01 allele.

The TCRs of the invention may provide any one or more of a variety of advantages, including when expressed by cells used for adoptive cell transfer. Without being bound to a particular theory or mechanism, it is believed that the inventive TCRs advantageously target the destruction of cancer cells while minimizing or eliminating the destruction of normal, non-cancerous cells, thereby reducing toxicity (which could be minimized or eliminated altogether). CD22 expression is limited to B-cell lineage lymphocytes and B-cell lineage precursors, and no other normal tissues express CD22. Therefore, the TCRs of the invention are not expected to cause any tissue damage to normal tissues and organs aside from B cells. Moreover, the inventive TCRs may, advantageously, successfully treat or prevent CD22-positive cancers that do not respond to other types of treatment such as, for example, chemotherapy, antibody therapies, surgery, or radiation. Additionally, the inventive TCRs may provide highly avid recognition of CD22, which may provide the ability to recognize unmanipulated tumor cells. Accordingly, the inventive TCRs may provide treatment options for CD22-expressing diseases that are resistant to existing treatments. Additionally, the inventive TCRs may overcome known resistance mechanisms to existing therapy seen in leukemia and lymphoma, mediating anti-leukemia/lymphoma responses even in the most refractory settings—known resistance mechanisms to the existing therapies involve loss of target antigen expression from the cell surface or loss of antibody epitope, despite protein expression being retained either partially and/or intracellularly. These resistance mechanisms do not affect TCR-mediated target recognition, because epitopes for TCRs are short fragment of peptides (usually 8-12-mer amino acids for MHC class I) processed intracellularly and presented in the context of MHC, and thus, TCRs do not require that target proteins to be expressed on the cell surface in a conformationally intact manner. The inventive TCRs have broad possible clinical indications as some B-cell malignancies originating from pre- or pro-B cells (acute lymphoblastic leukemia) express CD22, and the inventive TCRs were isolated from human repertoire. Moreover, in aspects, the inventive TCRs, polypeptides and proteins may comprise human amino acid sequences, which may reduce the risk of rejection by the human immune system as compared to, e.g., TCRs, polypeptides and proteins comprising only mouse amino acid sequences. The number of patients who may benefit from treatment in the long term could be as high as 30,000 new patients per year in the U.S. (this calculation is based on approximately 75,000 newly-diagnosed B-lymphoid malignancies per year reported in the SEER database, 40% of which are HLA-A*02:01+).

The phrase “antigenic specificity,” as used herein, means that the TCR can specifically bind to and immunologically recognize CD22 with high avidity. For example, a TCR may be considered to have “antigenic specificity” for CD22 if about 1×10⁴ to about 1×10⁵ T cells expressing the TCR secrete at least about 200 pg/mL or more (e.g., 200 pg/mL or more, 300 pg/mL or more, 400 pg/mL or more, 500 pg/mL or more, 600 pg/mL or more, 700 pg/mL or more, 1000 pg/mL or more, 5,000 pg/mL or more, 7,000 pg/mL or more, 10,000 pg/mL or more, 20,000 pg/mL or more, or a range defined by any two of the foregoing values) of IFNγ upon co-culture with (a) antigen-negative, HLA Class I molecule positive target cells pulsed with a low concentration of CD22 peptide (e.g., about 0.05 ng/mL to about 10 ng/mL, 1 ng/mL, 2 ng/mL, 5 ng/mL, 8 ng/mL, 10 ng/mL, or a range defined by any two of the foregoing values) or (b) antigen-negative, HLA Class I molecule positive target cells into which a nucleotide sequence encoding CD22 has been introduced such that the target cell expresses CD22, or (c) HLA Class I molecule positive target cells that naturally express CD22. Cells expressing the inventive TCRs may also secrete IFNγ upon co-culture with antigen-negative, HLA Class I molecule positive target cells pulsed with higher concentrations of CD22 peptide. The HLA Class I molecule may be any of the HLA Class I molecules described herein (e.g., an HLA-A*02:01 molecule).

Alternatively or additionally, a TCR may be considered to have “antigenic specificity” for CD22 if T cells expressing the TCR secrete at least twice as much IFNγ upon co-culture with (a) antigen-negative, HLA Class I molecule positive target cells pulsed with a low concentration of CD22 peptide or (b) antigen-negative, HLA Class I molecule positive target cells into which a nucleotide sequence encoding CD22 has been introduced such that the target cell expresses CD22 or (c) HLA Class I molecule positive target cells that naturally express CD22 as compared to the amount of IFNγ measured in a negative control. The negative control may be, for example, (i) T cells expressing the TCR, co-cultured with (a) antigen-negative, HLA Class I molecule positive target cells pulsed with the same concentration of an irrelevant peptide (e.g., some other peptide with a different sequence from the CD22 peptide) or (b) antigen-negative, HLA Class I molecule positive target cells into which a nucleotide sequence encoding an irrelevant protein/peptide has been introduced such that the target cell expresses the irrelevant protein/peptide, or (ii) untransduced T cells (e.g., derived from PBMC, which do not express the TCR) co-cultured with (a) antigen-negative, HLA Class I molecule positive target cells pulsed with the same concentration of CD22 peptide or (b) antigen-negative, HLA Class I molecule positive target cells into which a nucleotide sequence encoding CD22 has been introduced such that the target cell expresses CD22 or (c) HLA Class I molecule positive target cells that naturally express CD22. The HLA Class I molecule expressed by the target cells of the negative control would be the same HLA Class I molecule expressed by the target cells that are co-cultured with the T cells being tested. The HLA Class I molecule may be any of the HLA Class I molecules described herein (e.g., an HLA-A*02:01 molecule). IFNγ secretion may be measured by methods known in the art such as, for example, enzyme-linked immunosorbent assay (ELISA).

Alternatively or additionally, a TCR may be considered to have “antigenic specificity” for CD22 if at least twice as many of the numbers of T cells expressing the TCR secrete IFNγ upon co-culture with (a) antigen-negative, HLA Class I molecule positive target cells pulsed with a low concentration of CD22 peptide or (b) antigen-negative, HLA Class I molecule positive target cells into which a nucleotide sequence encoding CD22 has been introduced such that the target cell expresses CD22 or (c) HLA Class I molecule positive target cells that naturally express CD22 as compared to the numbers of negative control T cells that secrete IFNγ. The HLA Class I molecule, concentration of peptide, and the negative control may be as described herein with respect to other aspects of the invention. The numbers of cells secreting IFNγ may be measured by methods known in the art such as, for example, ELISPOT.

Alternatively or additionally, a TCR may be considered to have “antigenic specificity” for CD22 if T cells expressing the TCR upregulate expression of one or more T-cell activation markers or cytokines as measured by, for example, flow cytometry after stimulation with target cells expressing CD22 and HLA Class I molecules. Examples of T-cell activation markers include 4-1BB, OX40, CD107a, CD69, and cytokines that are upregulated upon antigen stimulation (e.g., tumor necrosis factor (TNF), interleukin (IL)-2, etc.).

An aspect of the invention provides a TCR comprising two polypeptides (i.e., polypeptide chains), such as an alpha (α) chain of a TCR, a beta (β) chain of a TCR, a gamma (γ) chain of a TCR, a delta (δ) chain of a TCR, or a combination thereof. The polypeptides of the inventive TCR can comprise any amino acid sequence, provided that the TCR has antigenic specificity for CD22. In some aspects, the TCR is non-naturally occurring.

In an aspect of the invention, the TCR comprises two polypeptide chains, each of which comprises a variable region comprising a complementarity determining region (CDR)1, a CDR2, and a CDR3 of a TCR. In an aspect of the invention, the TCR comprises a first polypeptide chain comprising a CDR1 comprising the amino acid sequence of SEQ ID NO: 1 (CDR1 of α chain), a CDR2 comprising the amino acid sequence of SEQ ID NO: 2 (CDR2 of α chain), and a CDR3 comprising the amino acid sequence of SEQ ID NO: 3 (CDR3 of α chain), and a second polypeptide chain comprising a CDR1 comprising the amino acid sequence of SEQ ID NO: 4 (CDR1 of β chain), a CDR2 comprising the amino acid sequence of SEQ ID NO: 5 (CDR2 of β chain), and a CDR3 comprising the amino acid sequence of SEQ ID NO: 6 (CDR3 of β chain).

The CDR3 of SEQ ID NOS: 3 or 6, i.e., of the α chain or β chain or both, may further comprise a cysteine immediately N-terminal to the first amino acid of the CDR or a phenylalanine immediately C-terminal to the final amino acid or both.

In this regard, the inventive TCR can comprise any one or more of the amino acid sequences selected from SEQ ID NOs: 1-6. In an aspect of the invention, the TCR comprises the amino acid sequences of (a) SEQ ID NOs: 1-3, (b) SEQ ID NOs: 4-6, or (c) SEQ ID NOs: 1-6.

In aspects of the invention, the TCR comprises an amino acid sequence of a variable region of a TCR comprising the CDRs set forth above. In this regard, the TCR can comprise the amino acid sequence of SEQ ID NO: 7 (variable region of α chain with N-terminal signal peptide); SEQ ID NO: 8 or 9 (variable region of β chain with N-terminal signal peptide); SEQ ID NO: 10 (variable region of (chain without N-terminal signal peptide); SEQ ID NO: 11 (variable region of β chain without N-terminal signal peptide); both of SEQ ID NOs: 7, and 8 or 9; or both of SEQ ID NOs: 10 and 11. In aspects, the TCR may comprise a human variable region, e.g., a human α chain variable region and a human β chain variable region.

The inventive TCRs may further comprise an α chain constant region and a β chain constant region. The constant region may be derived from any suitable species such as, e.g., human or mouse. In aspects of the invention, the TCRs further comprise murine α and β chain constant regions or human α and β chain constant regions. As used herein, the term “murine” or “human,” when referring to a TCR or any component of α TCR described herein (e.g., complementarity determining region (CDR), variable region, constant region, a chain, and/or β chain), means a TCR (or component thereof) which is derived from a mouse or a human, respectively, i.e., a TCR (or component thereof) that originated from or was, at one time, expressed by a mouse T cell or a human T cell, respectively.

An aspect of the invention provides a chimeric TCR comprising a human variable region and a murine constant region, wherein the TCR has antigenic specificity for a CD22 amino acid sequence presented by an HLA Class I molecule. The murine constant region may provide any one or more advantages. For example, the murine constant region may diminish mispairing of the inventive TCR with the endogenous TCRs of the host cell into which the inventive TCR is introduced. Alternatively or additionally, the murine constant region may increase expression of the inventive TCR. The chimeric TCR may comprise the amino acid sequence of SEQ ID NO: 13 (wild-type (WT) murine α chain constant region), SEQ ID NO: 14 (WT murine β chain constant region), or both SEQ ID NOs: 13 and 14. Preferably, the inventive TCR comprises the amino acid sequences of both of SEQ ID NOs: 13 and 14. The chimeric TCR may comprise any of the murine constant regions described herein in combination with any of the CDR regions as described herein with respect to other aspects of the invention. In this regard, the TCR may comprise the amino acid sequences of (a) SEQ ID NOs: 1-3 and 13, (b) SEQ ID NOs: 4-6 and 14, or (c) SEQ ID NOs: 1-6 and 13 and 14.

In another aspect of the invention, the chimeric TCR may comprise any of the murine constant regions described herein in combination with any of the variable regions described herein with respect to other aspects of the invention. In this regard, the TCR may comprise the amino acid sequences of (a) both of SEQ ID NOS: 7 and 13; (b) both of SEQ ID NOS: 8 or 9, and 14; or (c) all of SEQ ID NOS: 7 and 13, and either of 8 or 9, and 14.

In another aspect of the invention, the TCR comprises the amino acid sequence(s) of SEQ ID NO: 15 (α chain with WT murine constant region and N-terminal signal peptide), SEQ ID NO: 16 or 17 (β chain with WT murine constant region and N-terminal signal peptide), SEQ ID NO: 18 (α chain with WT murine constant region and without N-terminal signal peptide), SEQ ID NO: 19 (β chain with WT murine constant region and without N-terminal signal peptide), both of SEQ ID NO: 15, and 16 or 17, or both of SEQ ID NOS: 18 and 19.

In aspects of the invention, the TCR comprises a substituted constant region. In this regard, the TCR may comprise the amino acid sequence of any of the TCRs described herein with one, two, three, or four amino acid substitution(s) in the constant region of one or both of the α and β chain. Preferably, the TCR comprises a murine constant region with one, two, three, or four amino acid substitution(s) in the murine constant region of one or both of the α and β chains. In an especially preferred aspect, the TCR comprises a murine constant region with one, two, three, or four amino acid substitution(s) in the murine constant region of the α chain and one amino acid substitution in the murine constant region of the β chain. In some aspects, the TCRs comprising the substituted constant region advantageously provide one or more of increased recognition of CD22⁺ targets, increased expression by a host cell, diminished mispairing with endogenous TCRs, and increased anti-tumor activity as compared to the parent TCR comprising an unsubstituted (wild-type) constant region. In general, the substituted amino acid sequences of the murine constant regions of the TCR α and β chains, SEQ ID NOs: 20 and 21, respectively, correspond with all or portions of the unsubstituted murine constant region amino acid sequences SEQ ID NOs: 13 and 14, respectively, with SEQ ID NO: 20 having one, two, three, or four amino acid substitution(s) when compared to SEQ ID NO: 13 and SEQ ID NO: 21 having one amino acid substitution when compared to SEQ ID NO: 14. In this regard, an aspect of the invention provides a TCR comprising the amino acid sequences of (a) SEQ ID NO: 20 (constant region of α chain), wherein (i) X at position 48 is Thr or Cys; (ii) X at position 112 is Ser, Ala, Val, Leu, Ile, Pro, Phe, Met, or Trp; (iii) X at position 114 is Met, Ala, Val, Leu, Ile, Pro, Phe, or Trp; and (iv) X at position 115 is Gly, Ala, Val, Leu, Ile, Pro, Phe, Met, or Trp; (b) SEQ ID NO: 21 (constant region of R chain), wherein X at position 57 is Ser or Cys; or (c) both of SEQ ID NOs: 20 and 21. In aspects of the invention, the TCR comprising SEQ ID NO: 20 does not comprise SEQ ID NO: 13 (unsubstituted murine constant region of α chain). In aspects of the invention, the TCR comprising SEQ ID NO: 21 does not comprise SEQ ID NO: 14 (unsubstituted murine constant region of β chain).

The first amino acid of any of the mouse alpha constant regions described herein may be different than N as provided in SEQ ID NOS: 13 and 20. For example, in any TCR construct, polypeptide, protein, etc., as described herein, this first amino acid can be encoded by a split codon (having nucleotides from both a variable region and a constant region) such that any of the murine alpha constant regions may have a different amino acid at that position. For example, there may be an H at the position corresponding to the first amino acid in the constant region. Similarly, first amino acid of any of the mouse beta constant regions described herein may be different than E as provided in SEQ ID NOS: 14 and 21, e.g., this first amino acid can be encoded by a split codon.

In aspects of the invention, the TCR comprises an α chain comprising a variable region and a constant region and a β chain comprising a variable region and a constant region. In this regard, the TCR may comprise (a) an α chain comprising the amino acid sequence of SEQ ID NO: 22 (α chain with N-terminal signal peptide), wherein: (i) X at position 180 of SEQ ID NO: 22 is Thr or Cys; (ii) X at position 244 of SEQ ID NO: 22 is Ser, Ala, Val, Leu, Ile, Pro, Phe, Met, or Trp; (iii) X at position 246 of SEQ ID NO: 22 is Met, Ala, Val, Leu, Ile, Pro, Phe, or Trp; and (iv) X at position 247 of SEQ ID NO: 22 is Gly, Ala, Val, Leu, Ile, Pro, Phe, Met, or Trp; (b) a β chain comprising the amino acid sequence of SEQ ID NO: 23 or 24 (0 chain with N-terminal signal peptide), wherein X at position 188 of SEQ ID NO: 23 or 24 is Ser or Cys; (c) both (a) and (b); (d) an α chain comprising the amino acid sequence of SEQ ID NO: 25 (α chain without N-terminal signal peptide), wherein: (i) X at position 159 of SEQ ID NO: 25 is Thr or Cys; (ii) X at position 223 of SEQ ID NO: 25 is Ser, Ala, Val, Leu, Ile, Pro, Phe, Met, or Trp; (iii) X at position 225 of SEQ ID NO: 25 is Met, Ala, Val, Leu, Ile, Pro, Phe, or Trp; and (iv) X at position 226 of SEQ ID NO: 25 is Gly, Ala, Val, Leu, Ile, Pro, Phe, Met, or Trp; (e) a β chain comprising the amino acid sequence of SEQ ID NO: 26 (D chain without N-terminal signal peptide), wherein X at position 169 of SEQ ID NO: 26 is Ser or Cys.

In aspects of the invention, the TCR comprising SEQ ID NO: 22 does not comprise SEQ ID NO: 15 (unsubstituted α chain). In aspects of the invention, the TCR comprising SEQ ID NO: 23 or 24 does not comprise SEQ ID NO: 16 or 17 (unsubstituted R chain). In aspects of the invention, the TCR comprising SEQ ID NO: 25 does not comprise SEQ ID NO: 18 (unsubstituted α chain). In aspects of the invention, the TCR comprising SEQ ID NO: 26 does not comprise SEQ ID NO: 19 (unsubstituted β chain).

In aspects of the invention, the substituted constant region includes cysteine substitutions in the constant region of one or both of the α and β chains to provide a cysteine-substituted TCR. Opposing cysteines in the α and the β chains provide a disulfide bond that links the constant regions of the α and the β chains of the substituted TCR to one another and which is not present in a TCR comprising the unsubstituted murine constant regions. In this regard, the TCR may be a cysteine-substituted TCR in which one or both of the native Thr at position 48 (Thr48) of SEQ ID NO: 13 and the native Ser at position 57 (Ser57) of SEQ ID NO: 14 may be substituted with Cys. Preferably, both of the native Thr48 of SEQ ID NO: 13 and the native Ser57 of SEQ ID NO: 14 are substituted with Cys. Examples of cysteine-substituted TCR constant regions sequences are set forth in Table 1. In aspects of the invention, the cysteine-substituted TCR comprises (i) SEQ ID NO: 20, (ii) SEQ ID NO: 21, or (iii) both of SEQ ID NOs: 20 and 21, wherein both of SEQ ID NOs: 20 and 21 are as defined in Table 1. The cysteine-substituted TCRs of the invention may include the substituted constant region in addition to any of the CDRs or variable regions described herein.

In aspects of the invention, the cysteine-substituted, chimeric TCR comprises a full length alpha chain and a full-length beta chain. Examples of cysteine-substituted, chimeric TCR alpha chain and beta chain sequences are set forth in Table 1. In aspects of the invention, the TCR comprises SEQ ID NO: 22; SEQ ID NO: 23 or 24; both of SEQ ID NO: 22, and 23 or 24; SEQ ID NO: 25; SEQ ID NO: 26; or both of SEQ ID NO: 25 and 26; wherein all of the sequences are as defined in Table 1.

TABLE 1

| SEQ ID NO: | Definitions of “X” in some aspects |
|---|---|
| SEQ ID NO: 20 | X at position 48 is Cys, |
| (constant region α chain) | X at position 112 is Ser, |
| | X at position 114 is Met, and |
| | X at position 115 is Gly. |
| SEQ ID NO: 21 | X at position 57 is Cys |
| (constant region β chain) | |
| SEQ ID NO: 22 | X at position 180 is Cys, |
| (TCR D146W8 α chain | X at position 244 is Ser, |
| with N-terminal signal | X at position 246 is Met, and |
| peptide) | X at position 247 is Gly. |
| SEQ ID NO: 23 or 24 | X at position 188 is Cys |
| (TCR D146W8 β chain | |
| with N-terminal signal | |
| peptide) | |
| SEQ ID NO: 25 | X at position 159 is Cys, |
| (TCR D146W8 α chain | X at position 223 is Ser, |
| without N-terminal signal | X at position 225 is Met, and |
| peptide) | X at position 226 is Gly. |
| SEQ ID NO: 26 | X at position 169 is Cys |
| (TCR D146W8 β chain | |
| without N-terminal signal | |
| peptide) | |

In aspects of the invention, the substituted amino acid sequence includes substitutions of one, two, or three amino acids in the transmembrane (TM) domain of the constant region of one or both of the α and 13 chains with a hydrophobic amino acid to provide a hydrophobic amino acid-substituted TCR (also referred to herein as an “LVL-modified TCR”). The hydrophobic amino acid substitution(s) in the TM domain of the TCR may increase the hydrophobicity of the TM domain of the TCR as compared to a TCR that lacks the hydrophobic amino acid substitution(s) in the TM domain. In this regard, the TCR is an LVL-modified TCR in which one, two, or three of the native Sen112, Met114, and Gly115 of SEQ ID NO: 13 may, independently, be substituted with Ala, Val, Leu, Ile, Pro, Phe, Met, or Trp; preferably with Leu, Ile, or Val. Preferably, all three of the native Ser112, Met114, and Gly115 of SEQ ID NO: 13 may, independently, be substituted with Ala, Val, Leu, Ile, Pro, Phe, Met, or Trp; preferably with Leu, Ile, or Val. In aspects of the invention, the LVL-modified TCR comprises (i) SEQ ID NO: 20, (ii) SEQ ID NO: 21, or (iii) both of SEQ ID NOs: 20 and 21, wherein both of SEQ ID NOs: 20 and 21 are as defined in Table 2. The LVL-modified TCRs of the invention may include the substituted constant region in addition to any of the CDRs or variable regions described herein.

In aspects of the invention, the LVL-modified TCR comprises a full length alpha chain and a full-length beta chain. Examples of LVL-modified TCR alpha chain and beta chain sequences are set forth in Table 2. In aspects of the invention, the LVL-modified TCR comprises SEQ ID NO: 22; SEQ ID NO: 23 or 24; both of SEQ ID NO: 22, and 23 or 24; SEQ ID NO: 25; SEQ ID NO: 26; or both of SEQ ID NO: 25 and 26; wherein all of the sequences are as defined in Table 2.

TABLE 2

| SEQ ID NO: | Definitions of “X” in some aspects |
|---|---|
| SEQ ID NO: 20 | X at position 48 is Thr; |
| (constant region α chain) | X at position 112 is Ser, Ala, Val, Leu, Ile, Pro, Phe, Met, or Trp; |
| | preferably wherein X at position 112 is Leu, Ile, or Val; |
| | especially preferably wherein X at position 112 is Leu; |
| | X at position 114 is Met, Ala, Val, Leu, Ile, Pro, Phe, or Trp; |
| | preferably wherein X at position 114 is Leu, Ile, or Val; |
| | especially preferably wherein X at position 114 is Ile; and |
| | X at position 115 is Gly, Ala, Val, Leu, Ile, Pro, Phe, Met, or Trp; |
| | preferably wherein X at position 115 is Leu, Ile, or Val; |
| | especially preferably wherein X at position 115 is Val; |
| | Wherein SEQ ID NO: 20 does not comprise SEQ ID NO: 13 |
| | (unsubstituted constant region of alpha chain) |
| SEQ ID NO: 21 | X at position 57 is Ser |
| (constant region β chain) | |
| SEQ ID NO: 22 | X at position 180 is Thr; |
| (TCR D146W8 α chain | X at position 244 is Ser, Ala, Val, Leu, Ile, Pro, Phe, Met, or Trp; |
| with N-terminal signal | preferably wherein X at position 244 is Leu, Ile, or Val; |
| peptide) | especially preferably wherein X at position 244 is Leu; |
| | X at position 246 is Met, Ala, Val, Leu, Ile, Pro, Phe, or Trp; |
| | preferably wherein X at position 246 is Leu, Ile, or Val; |
| | especially preferably wherein X at position 246 is Ile; and |
| | X at position 247 is Gly, Ala, Val, Leu, Ile, Pro, Phe, Met, or Trp; |
| | preferably wherein X at position 247 is Leu, Ile, or Val; |
| | especially preferably wherein X at position 247 is Val, |
| | Wherein SEQ ID NO: 22 does not comprise SEQ ID NO: 15 |
| | (unsubstituted alpha chain) |
| SEQ ID NO: 23 or 24 | X at position 188 is Ser |
| (TCR D146W8 β chain | |
| with N-terminal signal | |
| peptide) | |
| SEQ ID NO: 25 | X at position 159 is Thr; |
| (TCR D146W8 α chain | X at position 223 is Ser, Ala, Val, Leu, Ile, Pro, Phe, Met, or Trp; |
| without N-terminal signal | preferably wherein X at position 223 is Leu, Ile, or Val; |
| peptide) | especially preferably wherein X at position 223 is Leu; |
| | X at position 225 is Met, Ala, Val, Leu, Ile, Pro, Phe, or Trp; |
| | preferably wherein X at position 225 is Leu, Ile, or Val; |
| | especially preferably wherein X at position 225 is Ile; and |
| | X at position 226 is Gly, Ala, Val, Leu, Ile, Pro, Phe, Met, or Trp; |
| | preferably wherein X at position 226 is Leu, Ile, or Val; |
| | especially preferably wherein X at position 226 is Val, |
| | Wherein SEQ ID NO: 25 does not comprise SEQ ID NO: 18 |
| | (unsubstituted alpha chain) |
| SEQ ID NO: 26 | X at position 169 is Ser |
| (TCR D146W8 β chain | |
| without N-terminal signal | |
| peptide) | |

In aspects of the invention, the substituted amino acid sequence includes the cysteine substitutions in the constant region of one or both of the α and β chains in combination with the substitution(s) of one, two, or three amino acids in the transmembrane (TM) domain of the constant region of one or both of the α and β chains with a hydrophobic amino acid (also referred to herein as “cysteine-substituted, LVL-modified TCR”). In this regard, the TCR is a cysteine-substituted, LVL-modified, chimeric TCR in which the native Thr48 of SEQ ID NO: 13 is substituted with Cys; one, two, or three of the native Ser112, Met114, and Gly115 of SEQ ID NO: 13 are, independently, substituted with Ala, Val, Leu, Ile, Pro, Phe, Met, or Trp; preferably with Leu, Ile, or Val; and the native Ser57 of SEQ ID NO: 14 is substituted with Cys. Preferably, all three of the native Ser112, Met114, and Gly115 of SEQ ID NO: 13 may, independently, be substituted with Ala, Val, Leu, Ile, Pro, Phe, Met, or Trp; preferably with Leu, Ile, or Val. In aspects of the invention, the cysteine-substituted, LVL-modified TCR comprises (i) SEQ ID NO: 20, (ii) SEQ ID NO: 21, or (iii) both of SEQ ID NOs: 20 and 21, wherein both of SEQ ID NOs: 20 and 21 are as defined in Table 3. The cysteine-substituted, LVL-modified TCRs of the invention may include the substituted constant region in addition to any of the CDRs or variable regions described herein.

In aspects, the cysteine-substituted, LVL-modified TCR comprises a full-length alpha chain and a full-length beta chain. In aspects of the invention, the LVL-modified TCR comprises SEQ ID NO: 22; SEQ ID NO: 23 or 24; both of SEQ ID NO: 22, and 23 or 24; SEQ ID NO: 25; SEQ ID NO: 26; both of SEQ ID NO: 25 and 26; wherein all of the sequences are as defined in Table 3.

TABLE 3

| SEQ ID NO: | Definitions of “X” in some aspects |
|---|---|
| SEQ ID NO: 20 | X at position 48 is Cys; |
| (constant region α | X at position 112 is Ser, Ala, Val, Leu, Ile, Pro, Phe, Met, or Trp; |
| chain) | preferably wherein X at position 112 is Leu, Ile, or Val; |
| | especially preferably wherein X at position 112 is Leu; |
| | X at position 114 is Met, Ala, Val, Leu, Ile, Pro, Phe, or Trp; |
| | preferably wherein X at position 114 is Leu, Ile, or Val; |
| | especially preferably wherein X at position 114 is Ile; and |
| | X at position 115 is Gly, Ala, Val, Leu, Ile, Pro, Phe, Met, or Trp; |
| | preferably wherein X at position 115 is Leu, Ile, or Val; and |
| | especially preferably wherein X at position 115 is Val, |
| | wherein SEQ ID NO: 20 does not simultaneously comprise all of Ser at |
| | position 112, Met at position 114, and Gly at position 115. |
| SEQ ID NO: 21 | X at position 57 is Cys |
| (constant region β | |
| chain) | |
| SEQ ID NO: 22 | X at position 180 is Cys; |
| (TCR D146W8 α chain | X at position 244 is Ser, Ala, Val, Leu, Ile, Pro, Phe, Met, or Trp; |
| with N-terminal signal | preferably wherein X at position 244 is Leu, Ile, or Val; |
| peptide) | especially preferably wherein X at position 244 is Leu; |
| | X at position 246 is Met, Ala, Val, Leu, Ile, Pro, Phe, or Trp; |
| | preferably wherein X at position 246 is Leu, Ile, or Val; |
| | especially preferably wherein X at position 246 is Ile; and |
| | X at position 247 is Gly, Ala, Val, Leu, Ile, Pro, Phe, Met, or Trp; |
| | preferably wherein X at position 247 is Leu, Ile, or Val; and |
| | especially preferably wherein X at position 247 is Val, |
| | wherein SEQ ID NO: 22 does not simultaneously comprise all of Ser at |
| | position 244, Met at position 246, and Gly at position 247. |
| SEQ ID NO: 23 or 24 | X at position 188 is Cys |
| (TCR D146W8 β chain | |
| with N-terminal signal | |
| peptide) | |
| SEQ ID NO: 25 | X at position 159 is Cys; |
| (TCR D146W8 α chain | X at position 223 is Ser, Ala, Val, Leu, Ile, Pro, Phe, Met, or Trp; |
| without N-terminal | preferably wherein X at position 223 is Leu, Ile, or Val; |
| signal peptide) | especially preferably wherein X at position 223 is Leu; |
| | X at position 225 is Met, Ala, Val, Leu, Ile, Pro, Phe, or Trp; |
| | preferably wherein X at position 225 is Leu, Ile, or Val; |
| | especially preferably wherein X at position 225 is Ile; and |
| | X at position 226 is Gly, Ala, Val, Leu, Ile, Pro, Phe, Met, or Trp; |
| | preferably wherein X at position 226 is Leu, Ile, or Val; and |
| | especially preferably wherein X at position 226 is Val, |
| | wherein SEQ ID NO: 25 does not simultaneously comprise all of Ser at |
| | position 223, Met at position 225, and Gly at position 226. |
| SEQ ID NO: 26 | X at position 169 is Cys |
| (TCR D146W8 β chain | |
| without N-terminal | |
| signal peptide) | |

Also provided by an aspect of the invention is a polypeptide comprising a functional portion of any of the TCRs described herein. The term “polypeptide,” as used herein, includes oligopeptides and refers to a single chain of amino acids connected by one or more peptide bonds.

With respect to the inventive polypeptides, the functional portion can be any portion comprising contiguous amino acids of the TCR of which it is a part, provided that the functional portion specifically binds to CD22. The term “functional portion,” when used in reference to a TCR, refers to any part or fragment of the TCR of the invention, which part or fragment retains the biological activity of the TCR of which it is a part (the parent TCR). Functional portions encompass, for example, those parts of a TCR that retain the ability to specifically bind to CD22 (e.g., within the context of an HLA-A*02:01 molecule), or detect, treat, or prevent cancer, to a similar extent, the same extent, or to a higher extent, as the parent TCR. In reference to the parent TCR, the functional portion can comprise, for instance, about 10%, about 25%, about 30%, about 50%, about 70%, about 80%, about 90%, about 95%, or more, of the parent TCR.

The functional portion can comprise additional amino acids at the amino or carboxy terminus of the portion, or at both termini, which additional amino acids are not found in the amino acid sequence of the parent TCR. Desirably, the additional amino acids do not interfere with the biological function of the functional portion, e.g., specifically binding to CD22; and/or having the ability to detect cancer, treat or prevent cancer, etc. More desirably, the additional amino acids enhance the biological activity, as compared to the biological activity of the parent TCR.

The polypeptide can comprise a functional portion of either or both of the α and β chains of the TCRs of the invention, such as a functional portion comprising one or more of the CDR1, CDR2, and CDR3 of the variable region(s) of the α chain and/or β chain of α TCR of the invention. In an aspect of the invention, the polypeptide can comprise the amino acid sequence of SEQ ID NO: 1 (CDR1 of α chain), SEQ ID NO: 2 (CDR2 of α chain), SEQ ID NO: 3 (CDR3 of α chain), SEQ ID NO: 4 (CDRT of β chain), SEQ ID NO: 5 (CDR2 of β chain), SEQ ID NO: 6 (CDR3 of β chain), or a combination thereof. The CDR3 of SEQ ID NO: 3 or 6, i.e., of the α chain or β chain or both, may further comprise a cysteine immediately N-terminal to the first amino acid of the CDR or a phenylalanine immediately C-terminal to the final amino acid or both.

In this regard, the inventive polypeptide can comprise any one or more of the amino acid sequences selected from SEQ ID NOs: 1-6. In an aspect of the invention, the TCR comprises the amino acid sequences of all of (a) SEQ ID NOs: 1-3, (b) SEQ ID NOs: 4-6, or (c) SEQ ID NOs: 1-6. In a preferred aspect, the polypeptide comprises the amino acid sequences of SEQ ID NOs: 1-6.

In an aspect of the invention, the inventive polypeptide can comprise, for instance, the variable region of the inventive TCR comprising a combination of the CDR regions set forth above. In this regard, the polypeptide can comprise the amino acid sequence of SEQ ID NO: 7 (variable region of α chain with N-terminal signal peptide); SEQ ID NO: 8 or 9 (variable region of β chain with N-terminal signal peptide); SEQ ID NO: 10 (variable region of α chain without N-terminal signal peptide); SEQ ID NO: 11 (variable region of β chain without N-terminal signal peptide); both of SEQ ID NOs: 7, and 8 or 9; or both of SEQ ID NOs: 10 and 11.

In aspects of the invention, the inventive polypeptide can further comprise the constant region of the inventive TCR set forth above. In this regard, the polypeptide can further comprise the amino acid sequence of SEQ ID NO: 13 (WT murine constant region of α chain), SEQ ID NO: 14 (WT murine constant region of β chain), SEQ ID NO: 20, (substituted murine constant region of α chain), SEQ ID NO: 21 (substituted murine constant region of β chain), both SEQ ID NOs: 13 and 14, or both SEQ ID NOs: 20 and 21. Preferably, the polypeptide further comprises the amino acid sequences of both of SEQ ID NOs: 13 and 14 or both of SEQ ID NO: 20 and 21 in combination with any of the CDR regions or variable regions described herein with respect to other aspects of the invention.

In aspects of the invention, the polypeptide comprises: (a) the amino acid sequence of SEQ ID NO: 20, wherein: (i) X at position 48 of SEQ ID NO: 20 is Thr or Cys; (ii) X at position 112 of SEQ ID NO: 20 is Ser, Ala, Val, Leu, Ile, Pro, Phe, Met, or Trp; (iii) X at position 114 of SEQ ID NO: 20 is Met, Ala, Val, Leu, Ile, Pro, Phe, or Trp; and (iv) X at position 115 of SEQ ID NO: 20 is Gly, Ala, Val, Leu, Ile, Pro, Phe, Met, or Trp; (b) the amino acid sequence of SEQ ID NO: 21, wherein X at position 57 of SEQ ID NO: 21 is Ser or Cys; or (c) both (a) and (b). In aspects of the invention, one or both of SEQ ID NOs: 20 and 21 of the polypeptide are as defined in any one of Tables 1-3.

In aspects of the invention, the inventive polypeptide can comprise the entire length of an α or β chain of the TCR described herein. In this regard, the inventive polypeptide can comprise the amino acid sequence of both of SEQ ID NO: 15, and 16 or 17; or both of SEQ ID NOS: 18 and 19. The polypeptide of the invention can comprise both chains of the TCRs described herein.

In aspects of the invention, the polypeptide comprises (a) an α chain comprising the amino acid sequence of SEQ ID NO: 22 (α chain with N-terminal signal peptide), wherein: (i) X at position 180 of SEQ ID NO: 22 is Thr or Cys; (ii) X at position 244 of SEQ ID NO: 22 is Ser, Ala, Val, Leu, Ile, Pro, Phe, Met, or Trp; (iii) X at position 246 of SEQ ID NO: 22 is Met, Ala, Val, Leu, Ile, Pro, Phe, or Trp; and (iv) X at position 247 of SEQ ID NO: 22 is Gly, Ala, Val, Leu, Ile, Pro, Phe, Met, or Trp; (b) a β chain comprising the amino acid sequence of SEQ ID NO: 23 or 24 (0 chain with N-terminal signal peptide), wherein X at position 188 of SEQ ID NO: 23 or 24 is Ser or Cys; (c) both (a) and (b); (d) an α chain comprising the amino acid sequence of SEQ ID NO: 25 (α chain without N-terminal signal peptide), wherein: (i) X at position 159 of SEQ ID NO: 25 is Thr or Cys; (ii) X at position 223 of SEQ ID NO: 25 is Ser, Ala, Val, Leu, Ile, Pro, Phe, Met, or Trp; (iii) X at position 225 of SEQ ID NO: 25 is Met, Ala, Val, Leu, Ile, Pro, Phe, or Trp; and (iv) X at position 226 of SEQ ID NO: 25 is Gly, Ala, Val, Leu, Ile, Pro, Phe, Met, or Trp; (e) a β chain comprising the amino acid sequence of SEQ ID NO: 26 (β chain without N-terminal signal peptide), wherein X at position 169 of SEQ ID NO: 26 is Ser or Cys; or (f) both (d) and (e). In an aspect of the invention, any one or more of the sequences of this paragraph comprising the polypeptide are as defined in any one of Tables 1-3.

An aspect of the invention further provides a protein comprising at least one of the polypeptides described herein. By “protein” is meant a molecule comprising one or more polypeptide chains.

In an aspect, the protein of the invention can comprise a first polypeptide chain comprising the amino acid sequences of SEQ ID NOs: 1-3 and a second polypeptide chain comprising the amino acid sequences of SEQ ID NOs: 4-6. The CDR3 of SEQ ID NO: 3 or 6, i.e., of the α chain or β chain or both, may further comprise a cysteine immediately N-terminal to the first amino acid of the CDR or a phenylalanine immediately C-terminal to the final amino acid or both.

In another aspect of the invention, the protein may comprise (i) a first polypeptide chain comprising the amino acid sequence of SEQ ID NO: 7 and a second polypeptide chain comprising the amino acid sequence of SEQ ID NO: 8 or 9; or (ii) a first polypeptide chain comprising the amino acid sequence of SEQ ID NO: 10 and a second polypeptide chain comprising the amino acid sequence of SEQ ID NO: 11.

The inventive protein may further comprise any of the constant regions described herein with respect to other aspects of the invention. In this regard, in aspects of the invention, the first polypeptide chain may further comprise the amino acid sequence of SEQ ID NO: 13 and the second polypeptide chain may further comprise the amino acid sequence of SEQ ID NO: 14. In aspects of the invention, the first polypeptide chain may further comprise the amino acid sequence of SEQ ID NO: 20 and the second polypeptide chain may further comprise the amino acid sequence of SEQ ID NO: 21.

In aspects of the invention, the protein comprises: (a) a first polypeptide chain comprising the amino acid sequence of SEQ ID NO: 20 (constant region of α chain), wherein (i) X at position 48 is Thr or Cys; (ii) X at position 112 is Ser, Ala, Val, Leu, Ile, Pro, Phe, Met, or Trp; (iii) X at position 114 is Met, Ala, Val, Leu, Ile, Pro, Phe, or Trp; and (iv) X at position 115 is Gly, Ala, Val, Leu, Ile, Pro, Phe, Met, or Trp; (b) a second polypeptide chain comprising the amino acid sequence of SEQ ID NO: 21 (constant region of β chain), wherein X at position 57 is Ser or Cys; or (c) both (a) and (b). In aspects of the invention, one or both of SEQ ID NOs: 20 and 21 of the protein are as defined in any one of Tables 1-3.

Alternatively or additionally, the protein of an aspect of the invention can comprise (a) a first polypeptide chain comprising the amino acid sequence of SEQ ID NO: 22 (α chain with N-terminal signal peptide), wherein: (i) X at position 180 of SEQ ID NO: 22 is Thr or Cys; (ii) X at position 244 of SEQ ID NO: 22 is Ser, Ala, Val, Leu, Ile, Pro, Phe, Met, or Trp; (iii) X at position 246 of SEQ ID NO: 22 is Met, Ala, Val, Leu, Ile, Pro, Phe, or Trp; and (iv) X at position 247 of SEQ ID NO: 22 is Gly, Ala, Val, Leu, Ile, Pro, Phe, Met, or Trp; (b) a second polypeptide chain comprising the amino acid sequence of SEQ ID NO: 23 or 24 (β chain with N-terminal signal peptide), wherein X at position 188 of SEQ ID NO: 23 or 24 is Ser or Cys; (c) both (a) and (b); (d) a first polypeptide chain comprising the amino acid sequence of SEQ ID NO: 25 (α chain without N-terminal signal peptide), wherein: (i) X at position 159 of SEQ ID NO: 25 is Thr or Cys; (ii) X at position 223 of SEQ ID NO: 25 is Ser, Ala, Val, Leu, Ile, Pro, Phe, Met, or Trp; (iii) X at position 225 of SEQ ID NO: 25 is Met, Ala, Val, Leu, Ile, Pro, Phe, or Trp; and (iv) X at position 226 of SEQ ID NO: 25 is Gly, Ala, Val, Leu, Ile, Pro, Phe, Met, or Trp; (e) a second polypeptide chain comprising the amino acid sequence of SEQ ID NO: 26 (0 chain without N-terminal signal peptide), wherein X at position 169 of SEQ ID NO: 26 is Ser or Cys; or (f) both (d) and (e). In an aspect of the invention, one or more of the sequences of this paragraph are as defined in any one of Tables 1-3.

The protein of the invention can be a TCR. Alternatively, if, for example, the protein comprises a single polypeptide chain or if the first and/or second polypeptide chain(s) of the protein further comprise(s) other amino acid sequences, e.g., an amino acid sequence encoding an immunoglobulin or a portion thereof, then the inventive protein can be a fusion protein. In this regard, an aspect of the invention also provides a fusion protein comprising at least one of the inventive polypeptides described herein along with at least one other polypeptide. The other polypeptide can exist as a separate polypeptide of the fusion protein, or can exist as a polypeptide, which is expressed in frame (in tandem) with one of the inventive polypeptides described herein. The other polypeptide can encode any peptidic or proteinaceous molecule, or a portion thereof, including, but not limited to an immunoglobulin, CD3, CD4, CD8, an MHC molecule, a CD1 molecule, e.g., CD1a, CD1b, CD1c, CD1d, etc.

The fusion protein can comprise one or more copies of the inventive polypeptide and/or one or more copies of the other polypeptide. For instance, the fusion protein can comprise 1, 2, 3, 4, 5, or more, copies of the inventive polypeptide and/or of the other polypeptide. Suitable methods of making fusion proteins are known in the art, and include, for example, recombinant methods.

In some aspects of the invention, the TCRs, polypeptides, and proteins of the invention may be expressed as a single protein comprising a linker peptide linking the α chain and the β chain. In this regard, the TCRs, polypeptides, and proteins of the invention may further comprise a linker peptide. The linker peptide may advantageously facilitate the expression of α recombinant TCR, polypeptide, and/or protein in a host cell. The linker peptide may comprise any suitable amino acid sequence. For example, the linker peptide may be a furin-SGSG-P2A linker comprising the amino acid sequence of RAKRSGSGATNFSLLKQAGDVEENPGP (SEQ ID NO: 27). Upon expression of the construct including the linker peptide by a host cell, the linker peptide may be cleaved, resulting in separated α and β chains. In aspects of the invention, the TCR, polypeptide, or protein may comprise an amino acid sequence comprising a full-length α chain, a full-length β chain, and a linker peptide positioned between the α and β chains, for example α chain-linker-β chain or β chain-linker-α chain. SEQ ID NO: 28 is β chain-linker-α chain.

The protein of the invention can be a recombinant antibody, or an antigen binding portion thereof, comprising at least one of the inventive polypeptides described herein. As used herein, “recombinant antibody” refers to a recombinant (e.g., genetically engineered) protein comprising at least one of the polypeptides of the invention and a polypeptide chain of an antibody, or an antigen binding portion thereof. The polypeptide of an antibody, or antigen binding portion thereof, can be a heavy chain, a light chain, a variable or constant region of a heavy or light chain, a single chain variable fragment (scFv), or an Fe, Fab, or F(ab)₂′ fragment of an antibody, etc. The polypeptide chain of an antibody, or an antigen binding portion thereof, can exist as a separate polypeptide of the recombinant antibody. Alternatively, the polypeptide chain of an antibody, or an antigen binding portion thereof, can exist as a polypeptide, which is expressed in frame (in tandem) with the polypeptide of the invention. The polypeptide of an antibody, or an antigen binding portion thereof, can be a polypeptide of any antibody or any antibody fragment, including any of the antibodies and antibody fragments described herein.

Included in the scope of the invention are functional variants of the inventive TCRs, polypeptides, or proteins described herein. The term “functional variant,” as used herein, refers to a TCR, polypeptide, or protein having substantial or significant sequence identity or similarity to a parent TCR, polypeptide, or protein, which functional variant retains the biological activity of the TCR, polypeptide, or protein of which it is a variant. Functional variants encompass, for example, those variants of the TCR, polypeptide, or protein described herein (the parent TCR, polypeptide, or protein) that retain the ability to specifically bind to CD22 for which the parent TCR has antigenic specificity or to which the parent polypeptide or protein specifically binds, to a similar extent, the same extent, or to a higher extent, as the parent TCR, polypeptide, or protein. In reference to the parent TCR, polypeptide, or protein, the functional variant can, for instance, be at least about 30%, about 50%, about 75%, about 80%, about 90%, about 95%, about 96%, about 97%, about 98%, about 99% or more identical in amino acid sequence to the parent TCR, polypeptide, or protein, respectively.

The functional variant can, for example, comprise the amino acid sequence of the parent TCR, polypeptide, or protein with at least one conservative amino acid substitution. Conservative amino acid substitutions are known in the art, and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

Alternatively or additionally, the functional variants can comprise the amino acid sequence of the parent TCR, polypeptide, or protein with at least one non-conservative amino acid substitution. In this case, it is preferable for the non-conservative amino acid substitution to not interfere with or inhibit the biological activity of the functional variant. Preferably, the non-conservative amino acid substitution enhances the biological activity of the functional variant, such that the biological activity of the functional variant is increased as compared to the parent TCR, polypeptide, or protein.

Each signal peptide of the TCRs, polypeptides, proteins, functional variants, and functional portions described herein, when present, can be any suitable TCR signal peptide, so long as the TCR, polypeptide, protein, or functional variant is expressed and has antigenic specificity for CD22 presented by an HLA Class I molecule.

The TCR, polypeptide, or protein can consist essentially of the specified amino acid sequence or sequences described herein, such that other components of the TCR, polypeptide, or protein, e.g., other amino acids, do not materially change the biological activity of the TCR, polypeptide, or protein. In this regard, the inventive TCR, polypeptide, or protein can, for example, consist essentially of the amino acid sequence of any of SEQ ID NOS: 15-19 or both of SEQ ID NO: 15, and 16 or 17, or both of SEQ ID NOS: 18 and 19.

The TCRs, polypeptides, and proteins of the invention can be of any length, i.e., can comprise any number of amino acids, provided that the TCRs, polypeptides, or proteins retain their biological activity, e.g., the ability to specifically bind to CD22; detect cancer in a mammal; or treat or prevent cancer in a mammal, etc. For example, the polypeptide can be in the range of from about 50 to about 5000 amino acids long, such as about 50, about 70, about 75, about 100, about 125, about 150, about 175, about 200, about 300, about 400, about 500, about 600, about 700, about 800, about 900, about 1000 or more amino acids in length. In this regard, the polypeptides of the invention also include oligopeptides.

The TCRs, polypeptides, and proteins of the invention can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

The TCRs, polypeptides, and proteins of the invention can be glycosylated, amidated, carboxylated, phosphorylated, esterified, N-acylated, cyclized via, e.g., a disulfide bridge, or converted into an acid addition salt and/or optionally dimerized or polymerized, or conjugated.

The TCR, polypeptide, and/or protein of the invention can be obtained by methods known in the art such as, for example, de novo synthesis. Also, polypeptides and proteins can be recombinantly produced using the nucleic acids described herein using standard recombinant methods. See, for instance, Green and Sambrook, Molecular Cloning: A Laboratory Manual, 4^{th} ed., Cold Spring Harbor Press, Cold Spring Harbor, NY (2012). Alternatively, the TCRs, polypeptides, and/or proteins described herein can be synthesized by any of a variety of commercial entities. In this respect, the inventive TCRs, polypeptides, and proteins can be synthetic, recombinant, isolated, and/or purified. An aspect of the invention provides an isolated or purified TCR, polypeptide, or protein encoded by any of the nucleic acids or vectors described herein with respect to other aspects of the invention. Another aspect of the invention provides an isolated or purified TCR, polypeptide, or protein that results from expression of any of the nucleic acids or vectors described herein with respect to other aspects of the invention in a cell. Still another aspect of the invention provides a method of producing any of the TCRs, polypeptides, or proteins described herein, the method comprising culturing any of the host cells or populations of host cells described herein so that the TCR, polypeptide, or protein is produced.

Included in the scope of the invention are conjugates, e.g., bioconjugates, comprising any of the inventive TCRs, polypeptides, or proteins (including any of the functional portions or variants thereof), nucleic acids, recombinant expression vectors, host cells, populations of host cells, or antibodies, or antigen binding portions thereof. Conjugates, as well as methods of synthesizing conjugates in general, are known in the art.

An aspect of the invention provides a nucleic acid comprising a nucleotide sequence encoding any of the TCRs, polypeptides, or proteins described herein. “Nucleic acid,” as used herein, includes “polynucleotide,” “oligonucleotide,” and “nucleic acid molecule,” and generally means a polymer of DNA or RNA, which can be single-stranded or double-stranded, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In aspects, the nucleic acid comprises complementary DNA (cDNA). It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

Preferably, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art. See, for example, Green and Sambrook et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from any of α variety of commercial entities.

The nucleic acid can comprise any nucleotide sequence which encodes any of the TCRs, polypeptides, or proteins described herein. The nucleic acids may include, for example, those listed in Table 4.

TABLE 4

| TCR | | SEQ ID |
|---|---|---|
| name | TCR chain | NO: |
| D146W8, | ATGAAATCCTTGAGAGTTTTACTAGTGATCCTGT | 29 |
| alpha | GGCTTCAGTTGAGCTGGGTTTGGAGCCAACAGAA | |
| chain | GGAGGTGGAGCAGAATTCTGGACCCCTCAGTGTT | |
| (VJ) | CCAGAGGGAGCCATTGCCTCTCTCAACTGCACTT | |
| | ACAGTGACCGAGGTTCCCAGTCCTTCTTCTGGTA | |
| | CAGACAATATTCTGGGAAAAGCCCTGAGTTGATA | |
| | ATGTTCATATACTCCAATGGTGACAAAGAAGATG | |
| | GAAGGTTTACAGCACAGCTCAATAAAGCCAGCCA | |
| | GTATGTTTCTCTGCTCATCAGAGACTCCCAGCCC | |
| | AGTGATTCAGCCACCTACCTCTGTGCCGTGAAAG | |
| | GTGCTGGAGGCTTCAAAACTATCTTTGGAGCAGG | |
| | AACAAGACTATTTGTTAAAGCAA | |
| D146W8, | ATGGGCACCAGCCTCCTCTGCTGGATGGCCCTGT | 30 |
| beta | GTCTCCTGGGGGCAGATCACGCAGATACTGGAGT | |
| chain | CTCCCAGAACCCCAGACACAAGATCACAAAGAGG | |
| (VDJ) | GGACAGAATGTAACTTTCAGGTGTGATCCAATTT | |
| | CTGAACACAACCGCCTTTATTGGTACCGACAGAC | |
| | CCTGGGGCAGGGCCCAGAGTTTCTGACTTACTTC | |
| | CAGAATGAAGCTCAACTAGAAAAATCAAGGCTGC | |
| | TCAGTGATCGGTTCTCTGCAGAGAGGCCTAAGGG | |
| | ATCTTTCTCCACCTTGGAGATCCAGCGCACAGAG | |
| | CAGGGGGACTCGGCCATGTATCTCTGTGCCAGCA | |
| | GCCCGGGTAACACTGAAGCTTTCTTTGGACAAGG | |
| | CACCAGACTCACAGTTGTAG | |

The amino acid between J and the constant region is encoded by a “split codon” one nucleotide from J and two nucleotides from constant region.

In aspects of the invention, the nucleic acid comprises a codon-optimized nucleotide sequence encoding any of the TCRs, polypeptides, or proteins described herein. Without being bound to any particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency.

The invention also provides a nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive TCRs. It is generally appreciated that conditions can be rendered more stringent by the addition of increasing amounts of formamide.

The invention also provides a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein. In this regard, the nucleic acid may consist essentially of any of the nucleotide sequences described herein.

An aspect of the invention provides an isolated or purified nucleic acid comprising, from 5′ to 3′, a first nucleic acid sequence and a second nucleotide sequence, wherein the first and second nucleotide sequence, respectively, encode the amino sequences of SEQ ID NOs: 7, and 8 or 9; 8 or 8, and 7; 10 and 11; or 11 and 10.

In an aspect of the invention, the isolated or purified nucleic acid further comprises a third nucleotide sequence interposed between the first and second nucleotide sequence, wherein the third nucleotide sequence encodes a cleavable linker peptide. In an aspect of the invention, the cleavable linker peptide comprises the amino acid sequence of SEQ ID NO: 27.

The nucleic acids of the invention can be incorporated into a recombinant expression vector. In this regard, the invention provides a recombinant expression vector comprising any of the nucleic acids of the invention. In aspects of the invention, the recombinant expression vector comprises a nucleotide sequence encoding the α chain, the R chain, and linker peptide.

For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotide, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages do not hinder the transcription or replication of the vector.

The recombinant expression vector of the invention can be any suitable recombinant expression vector, and can be used to transform or transfect any suitable host cell. Suitable vectors include those designed for propagation and expansion or for expression or both, such as plasmids and viruses. The vector can be selected from the pUC series (Fermentas Life Sciences), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA). Bacteriophage vectors, such as λGT10, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-C1, pMAM and pMAMneo (Clontech). Preferably, the recombinant expression vector is a viral vector, e.g., a retroviral vector. In an especially preferred aspect, the recombinant expression vector is an MSGV1 vector. In an aspect of the invention, the recombinant expression vector is a transposon or a lentiviral vector.

The recombinant expression vectors of the invention can be prepared using standard recombinant DNA techniques described in, for example, Green and Sambrook et al., supra. Constructs of expression vectors, which are circular or linear, can be prepared to contain a replication system functional in a prokaryotic or eukaryotic host cell. Replication systems can be derived, e.g., from ColE1, 2 μ plasmid, λ, SV40, bovine papillomavirus, and the like.

Desirably, the recombinant expression vector comprises regulatory sequences, such as transcription and translation initiation and termination codons, which are specific to the type of host cell (e.g., bacterium, fungus, plant, or animal) into which the vector is to be introduced, as appropriate and taking into consideration whether the vector is DNA- or RNA-based.

The recombinant expression vector can include one or more marker genes, which allow for selection of transformed or transfected host cells. Marker genes include biocide resistance, e.g., resistance to antibiotics, heavy metals, etc., complementation in an auxotrophic host cell to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the TCR, polypeptide, or protein, or to the nucleotide sequence which is complementary to or which hybridizes to the nucleotide sequence encoding the TCR, polypeptide, or protein. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, nitroreductase, and the inducible caspase 9 gene system.

Another aspect of the invention further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human or mouse. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the recombinant expression vector, the host cell is preferably a prokaryotic cell, e.g., a DH5α cell. For purposes of producing a recombinant TCR, polypeptide, or protein, the host cell is preferably a mammalian cell. Most preferably, the host cell is a human cell. While the host cell can be of any cell type, can originate from any type of tissue, and can be of any developmental stage, the host cell preferably is a peripheral blood lymphocyte (PBL) or a peripheral blood mononuclear cell (PBMC). More preferably, the host cell is a T cell. In an aspect of the invention, the host cell is a human lymphocyte. In another aspect of the invention, the host cell is selected from a T cell, a natural killer T (NKT) cell, an invariant natural killer T (iNKT) cell, and a natural killer (NK) cell. Still another aspect of the invention provides a method of producing a host cell expressing a TCR that has antigenic specificity for a CD22 amino acid sequence, e.g., the peptide of SEQ ID NO: 12, the method comprising contacting a cell with any of the vectors described herein under conditions that allow introduction of the vector into the cell.

For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. Preferably, the T cell is a human T cell. The T cell can be any type of T cell and can be of any developmental stage, including but not limited to, CD4⁺/CD8⁺ double positive T cells, CD4⁺ helper T cells, e.g., Th₁ and Th₂ cells, CD4⁺ T cells, CD8⁺ T cells (e.g., cytotoxic T cells), tumor infiltrating lymphocytes (TILs), memory T cells (e.g., central memory T cells and effector memory T cells), naïve T cells, and the like.

Also provided by the invention is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of α single host cell comprising a recombinant expression vector, such that all cells of the population comprise the recombinant expression vector. In one aspect of the invention, the population of cells is a clonal population comprising host cells comprising a recombinant expression vector as described herein.

In aspects of the invention, the numbers of cells in the population may be rapidly expanded. Expansion of the numbers of T cells can be accomplished by any of a number of methods as are known in the art as described in, for example, U.S. Pat. Nos. 8,034,334; 8,383,099; U.S. Patent Application Publication No. 2012/0244133; Dudley et al., J Immunother., 26:332-42 (2003); and Riddell et al., J. Immunol. Methods, 128:189-201 (1990). In aspects, expansion of the numbers of T cells is carried out by culturing the T cells with OKT3 antibody, IL-2, and feeder PBMC (e.g., irradiated allogeneic PBMC).

The inventive TCRs, polypeptides, proteins, nucleic acids, recombinant expression vectors, and host cells (including populations thereof), can be isolated and/or purified. The term “isolated,” as used herein, means having been removed from its natural environment. The term “purified,” as used herein, means having been increased in purity, wherein “purity” is a relative term, and not to be necessarily construed as absolute purity. For example, the purity can be at least about 50%, can be greater than about 60%, about 70%, about 80%, about 90%, about 95%, or can be about 100%.

The inventive TCRs, polypeptides, proteins, nucleic acids, recombinant expression vectors, and host cells (including populations thereof), all of which are collectively referred to as “inventive TCR materials” hereinafter, can be formulated into a composition, such as a pharmaceutical composition. In this regard, the invention provides a pharmaceutical composition comprising any of the TCRs, polypeptides, proteins, nucleic acids, expression vectors, and host cells (including populations thereof), described herein, and a pharmaceutically acceptable carrier. The inventive pharmaceutical compositions containing any of the inventive TCR materials can comprise more than one inventive TCR material, e.g., a polypeptide and a nucleic acid, or two or more different TCRs. Alternatively, the pharmaceutical composition can comprise an inventive TCR material in combination with another pharmaceutically active agent(s) or drug(s), such as a chemotherapeutic agent, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

Preferably, the carrier is a pharmaceutically acceptable carrier. With respect to pharmaceutical compositions, the carrier can be any of those conventionally used for the particular inventive TCR material under consideration. Methods for preparing administrable compositions are known or apparent to those skilled in the art and are described in more detail in, for example, Remington: The Science and Practice of Pharmacy, 22^{nd} Ed., Pharmaceutical Press (2012). It is preferred that the pharmaceutically acceptable carrier be one which has no detrimental side effects or toxicity under the conditions of use.

The choice of carrier will be determined in part by the particular inventive TCR material, as well as by the particular method used to administer the inventive TCR material. Accordingly, there are a variety of suitable formulations of the pharmaceutical composition of the invention. Suitable formulations may include any of those for parenteral, subcutaneous, intravenous, intramuscular, intraarterial, intrathecal, intratumoral, or interperitoneal administration. More than one route can be used to administer the inventive TCR materials, and in certain instances, a particular route can provide a more immediate and more effective response than another route.

Preferably, the inventive TCR material is administered by injection, e.g., intravenously. When the inventive TCR material is a host cell (or population thereof) expressing the inventive TCR, the pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, IL), PLASMA-LYTE A (Baxter, Deerfield, IL), about 5% dextrose in water, or Ringer's lactate. In aspects, the pharmaceutically acceptable carrier is supplemented with human serum albumin.

For purposes of the invention, the amount or dose (e.g., numbers of cells when the inventive TCR material is one or more cells) of the inventive TCR material administered should be sufficient to effect, e.g., a therapeutic or prophylactic response, in the subject or animal over a reasonable time frame. For example, the dose of the inventive TCR material should be sufficient to bind to a cancer antigen (e.g., CD22), or detect, treat or prevent cancer in a period of from about 2 hours or longer, e.g., 12 to 24 or more hours, from the time of administration. In certain aspects, the time period could be even longer. The dose will be determined by the efficacy of the particular inventive TCR material and the condition of the animal (e.g., human), as well as the body weight of the animal (e.g., human) to be treated.

Many assays for determining an administered dose are known in the art. For purposes of the invention, an assay, which comprises comparing the extent to which target cells are lysed or IFNγ is secreted by T cells expressing the inventive TCR, polypeptide, or protein upon administration of a given dose of such T cells to a mammal among a set of mammals of which each is given a different dose of the T cells, could be used to determine a starting dose to be administered to a mammal. The extent to which target cells are lysed or IFNγ is secreted upon administration of a certain dose can be assayed by methods known in the art.

The dose of the inventive TCR material also will be determined by the existence, nature and extent of any adverse side effects that might accompany the administration of a particular inventive TCR material. Typically, the attending physician will decide the dosage of the inventive TCR material with which to treat each individual patient, taking into consideration a variety of factors, such as age, body weight, general health, diet, sex, inventive TCR material to be administered, route of administration, and the severity of the cancer being treated. In aspects in which the inventive TCR material is a population of cells, the number of cells administered per infusion may vary, e.g., from about 1×10⁶ to about 1×10¹² cells or more. In certain aspects, fewer than 1×10⁶ cells may be administered.

One of ordinary skill in the art will readily appreciate that the inventive TCR materials of the invention can be modified in any number of ways, such that the therapeutic or prophylactic efficacy of the inventive TCR materials is increased through the modification. For instance, the inventive TCR materials can be conjugated either directly or indirectly through a bridge to a chemotherapeutic agent. The practice of conjugating compounds to a chemotherapeutic agent is known in the art. One of ordinary skill in the art recognizes that sites on the inventive TCR materials, which are not necessary for the function of the inventive TCR materials, are suitable sites for attaching a bridge and/or a chemotherapeutic agent, provided that the bridge and/or chemotherapeutic agent, once attached to the inventive TCR materials, do(es) not interfere with the function of the inventive TCR materials, i.e., the ability to bind to CD22 or to detect, treat, or prevent cancer.

It is contemplated that the inventive pharmaceutical compositions, TCRs, polypeptides, proteins, nucleic acids, recombinant expression vectors, host cells, and populations of cells can be used in methods of treating or preventing cancer. Without being bound to a particular theory, the inventive TCRs are believed to bind specifically to CD22, such that the TCR (or related inventive polypeptide or protein), when expressed by a cell, is able to mediate an immune response against a target cell expressing CD22. In this regard, the invention provides a method of treating or preventing cancer in a mammal, comprising administering to the mammal any of the pharmaceutical compositions, TCRs, polypeptides, or proteins described herein, any nucleic acid or recombinant expression vector comprising a nucleotide sequence encoding any of the TCRs, polypeptides, proteins described herein, or any host cell or population of cells comprising a recombinant vector which encodes any of the TCRs, polypeptides, or proteins described herein, in an amount effective to treat or prevent cancer in the mammal.

An aspect of the invention provides a method of inducing an immune response against a cancer in a mammal, comprising administering to the mammal any of the pharmaceutical compositions, TCRs, polypeptides, or proteins described herein, any nucleic acid or recombinant expression vector comprising a nucleotide sequence encoding any of the TCRs, polypeptides, or proteins described herein, or any host cell or population of cells comprising a recombinant vector which encodes any of the TCRs, polypeptides, or proteins described herein, in an amount effective to induce an immune response against the cancer in the mammal.

An aspect of the invention provides any of the pharmaceutical compositions, TCRs, polypeptides, or proteins described herein, any nucleic acid or recombinant expression vector comprising a nucleotide sequence encoding any of the TCRs, polypeptides, proteins described herein, or any host cell or population of cells comprising a recombinant vector which encodes any of the TCRs, polypeptides, or proteins described herein, for use in the treatment or prevention of cancer in a mammal.

An aspect of the invention provides any of the pharmaceutical compositions, TCRs, polypeptides, or proteins described herein, any nucleic acid or recombinant expression vector comprising a nucleotide sequence encoding any of the TCRs, polypeptides, or proteins described herein, or any host cell or population of cells comprising a recombinant vector which encodes any of the TCRs, polypeptides, or proteins described herein, for use in inducing an immune response against a cancer in a mammal.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the cancer being treated or prevented. For example, treatment or prevention can include promoting the regression of α tumor. Also, for purposes herein, “prevention” can encompass delaying the onset of the cancer, or a symptom or condition thereof. Alternatively or additionally, “prevention” may encompass preventing or delaying the recurrence of cancer, or a symptom or condition thereof.

Also provided is a method of detecting the presence of cancer in a mammal. The method comprises (i) contacting a sample comprising one or more cells from the mammal with any of the inventive TCRs, polypeptides, proteins, nucleic acids, recombinant expression vectors, host cells, populations of cells, or pharmaceutical compositions described herein, thereby forming a complex, and (ii) detecting the complex, wherein detection of the complex is indicative of the presence of cancer in the mammal.

With respect to the inventive method of detecting cancer in a mammal, the sample of cells can be a sample comprising whole cells, lysates thereof, or a fraction of the whole cell lysates, e.g., a nuclear or cytoplasmic fraction, a whole protein fraction, or a nucleic acid fraction.

For purposes of the inventive method of detecting cancer, the contacting can take place in vitro or in vivo with respect to the mammal. Preferably, the contacting is in vitro.

Also, detection of the complex can occur through any number of ways known in the art. For instance, the inventive TCRs, polypeptides, proteins, nucleic acids, recombinant expression vectors, host cells, or populations of cells, described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

For purposes of the inventive methods, wherein host cells or populations of cells are administered, the cells can be cells that are allogeneic or autologous to the mammal. Preferably, the cells are autologous to the mammal.

With respect to the inventive methods, the cancer can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, chronic lymphocytic leukemia, chronic myeloid cancer, colon cancer, colorectal cancer, endometrial cancer, esophageal cancer, uterine cervical cancer, gastrointestinal carcinoid tumor, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, and urinary bladder cancer. A preferred cancer is a leukemia or a lymphoma (such as Hodgkin lymphoma, T/NK cell lymphoma, or post-transplant lymphoproliferative disorders). In aspects of the invention, the cancer expresses a CD22 amino acid sequence, for example, the CD22 amino acid sequence is SEQ ID NO: 12. The CD22 expressed by the cancer may be as described herein with respect to other aspects of the invention. Treatment may also apply to other non-cancerous diseases where depletion of CD22-expression B cells have been shown to be efficacious, such as rheumatoid arthritis and other autoimmune diseases.

The mammal referred to in the inventive methods can be any mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

It shall be noted that the preceding are merely examples of aspects. Other exemplary aspects are apparent from the entirety of the description herein. It will also be understood by one of ordinary skill in the art that each of these aspects may be used in various combinations with the other aspects provided herein.

The following examples further illustrate the invention but, of course, should not be construed as in any way limiting its scope.

### Example 1

This Example demonstrates the method of identification of α TCR having antigenic specificity for CD22 p228-236 using allogeneic stimulation, in accordance with aspects of the invention.

CD22 p228-236 is reported to be naturally processed and presented in the context of HLA-A2 by normal B lymphocytes (Hassan et al., Mol. Cell Proteomics, 12(7): 1829-1843 (2013), incorporated by reference herein).

CD22 p228-236 is predicted to bind to the HLA-A*02:01 molecule with high affinity (Table 5). The MHC-I binding predictions were made using the IEDB analysis resource Consensus tool (Kim et al., Nucleic Acids Res., 40: W525-30 (2012), incorporated by reference herein) which combines predictions from ANN aka NetMHC (Nielsen et al., Protein Sci., 12: 1007-1017 (2003); Lundegaard et al., Nucleic Acids Res., 36: W509-512 (2008); Andreatta et al., Bioinformatics, 32:511-7 (2016), incorporated by reference herein), SMM (Peters et al., BMC Bioinformatics, 6: 132 (2005), incorporated by reference herein) and Comblib (Sidney et al., Immunome Res., 4:2 (2008), incorporated by reference herein).

TABLE 5

| | | | Proteasome | TAP | MHC | Processing | Total | MHC |
|---|---|---|---|---|---|---|---|---|
| Start | End | Peptide | Score | Score | Score | Score | Score | IC₅₀ |
| 228 | 236 | FLSNDTVQL | 1.64 | 0.43 | −0.76 | 2.06 | 1.3 | 5.8 |
| | | (SEQ ID NO: 12) | | | | | | |
| 8 | 16 | LLLLVLEYL | 1.56 | 0.41 | −1.35 | 1.97 | 0.62 | 22.3 |
| | | (SEQ ID NO: 31) | | | | | | |
| 3 | 11 | LLGPWLLLL | 1.49 | 0.35 | −1.34 | 1.84 | 0.5 | 21.8 |
| | | (SEQ ID NO: 32) | | | | | | |
| 2 | 11 | HLLGPWLLLL | 1.49 | 0.41 | −1.4 | 1.9 | 0.5 | 25.4 |
| | | (SEQ ID NO: 33) | | | | | | |
| 351 | 360 | FLCMSLANPL | 1.2 | 0.32 | −1.05 | 1.51 | 0.46 | 11.2 |
| | | (SEQ ID NO: 34) | | | | | | |

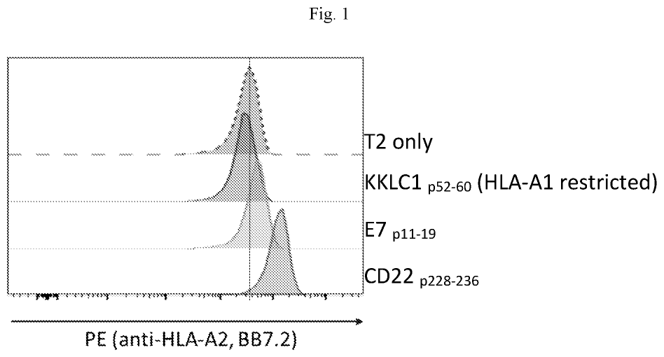

High affinity of CD22 p228-236 with HLA-A2 is supported by the T2 peptide-binding assay. T2 cells were incubated overnight in the absence or presence of peptide (KKLC1 p52-60, E7 p11-19, or CD22 p228-236) at 100 μg/mL. Surface HLA-A2 expression was assessed using flow cytometry analysis. The results are shown in FIG. 1 and Table 6.

TABLE 6

| | Mean Fluorescence | Predicted HLA- |
|---|---|---|
| | Intensity | A*02:01 IC₅₀ [nM] |
| T2 only | 3.34E+05 | — |
| KKLC1 ₚ₅₂₋₆₀ (HLA-A1 | 3.05E+05 | — |
| restricted control) | | |
| E7 ₚ₁₁₋₁₉ | 4.92E+05 | 30.4 |
| CD22 ₚ₂₂₈₋₂₃₆ | 1.29E+06 | 5.8 |

Up-regulation of HLA-A2 expression suggests that the peptide bound to the HLA-A2 and stabilized surface HLA-A2 expression.

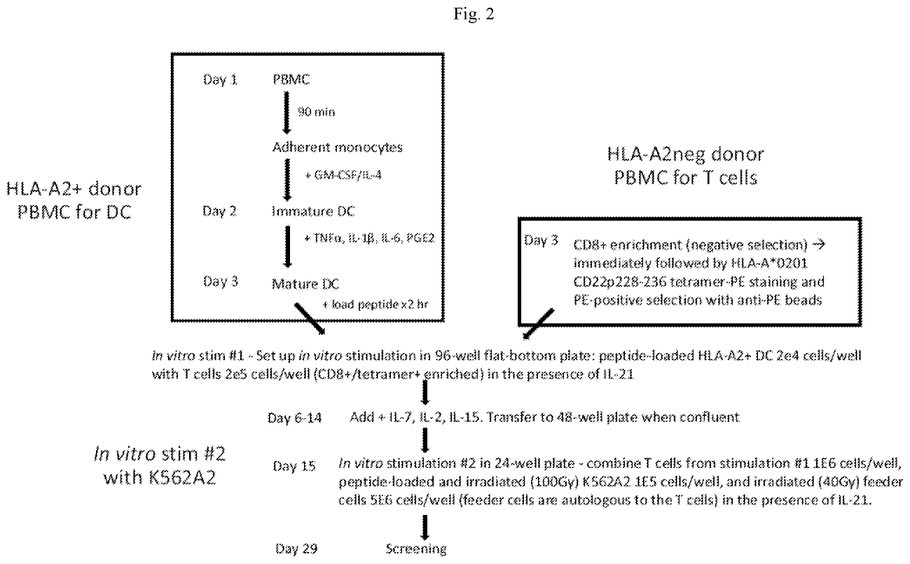

Screening of an HLA-A2-negative donor TCR repertoire was performed using an allogeneic TCR repertoire screening method (see FIG. 2).

An HLA-A2 negative donor's CD8+ T cells were first incubated with HLA-A2-peptide tetramer (for the HLA-A2-restricted epitope of interest), and tetramer-bound population was enriched with a positive selection (magnetic isolation). Tetramer+ population was then stimulated with allogeneic HLA-A2+ dendritic cells loaded with an epitope of interest in the presence of IL-21, then with IL-7, IL-2, and IL-15. Tetramer+ population was stimulated again, this time with an artificial APC derived from K562 that expressed only HLA-A*02:01 and no other HLA class I or class II alleles (named K562A2). Irradiated K562A2 cells loaded with an epitope peptide of interest were added to the co-culture wells. Approximately one month after the initial stimulation, co-culture wells were screened for the presence of TCRs with desired specificity.

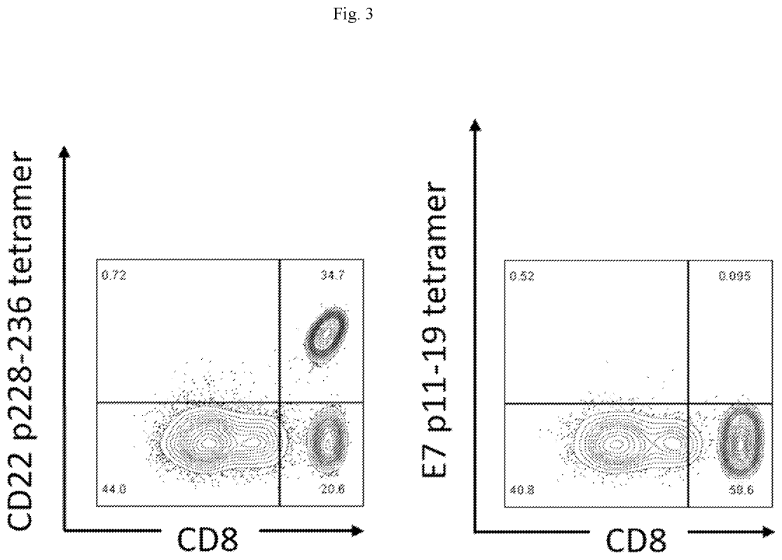

HLA-A*02:01 CD22 p228-236 tetramer-binding CD8+ T cells can be enriched from A2-PBMCs by repetitively stimulating them with A2+ DC/K562A2 loaded with CD22 p228-236. HLA-A*02:01 CD22 p228-236 tetramer-binding CD8+ T cells raised by this method are shown in FIG. 3, which demonstrates that such CD8 T cells bind specifically to CD22 p228-236 p-MHC tetramers but not to irrelevant tetramers. Tetramers were synthesized using UV peptide exchange technique (BioLegend Flex-T HLA-A*02:01 monomer). Cells were stained with antibodies and tetramer at 2-8° C. in dark for 30 min, followed by FACS (gated on singlet/live/lymphocytes).

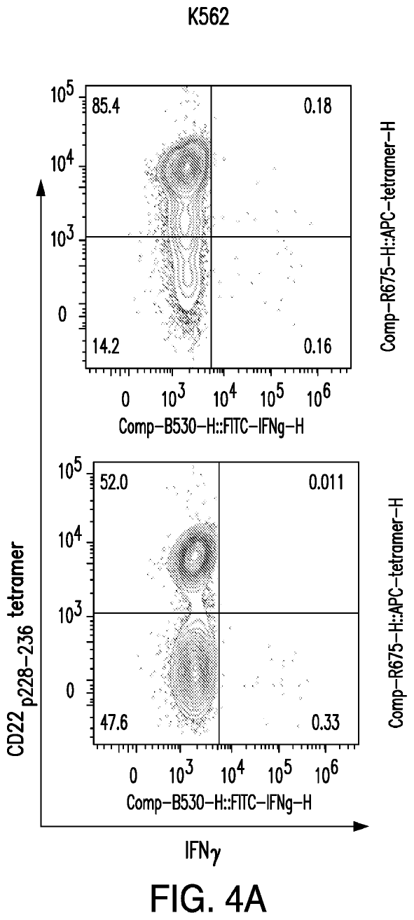

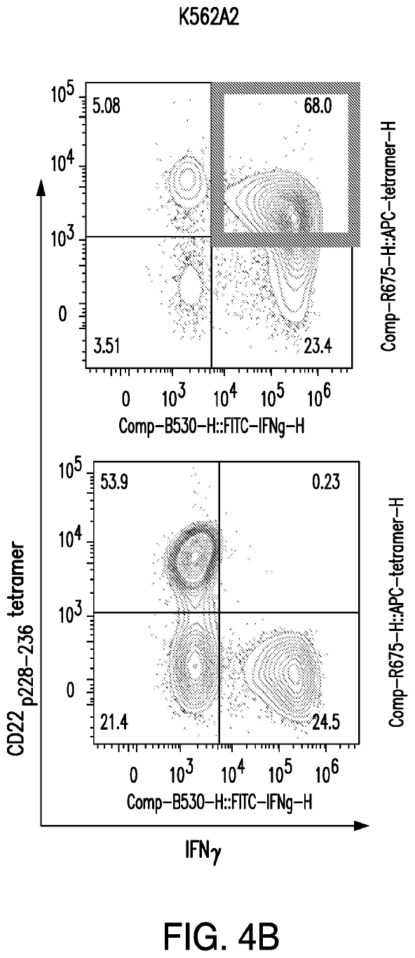

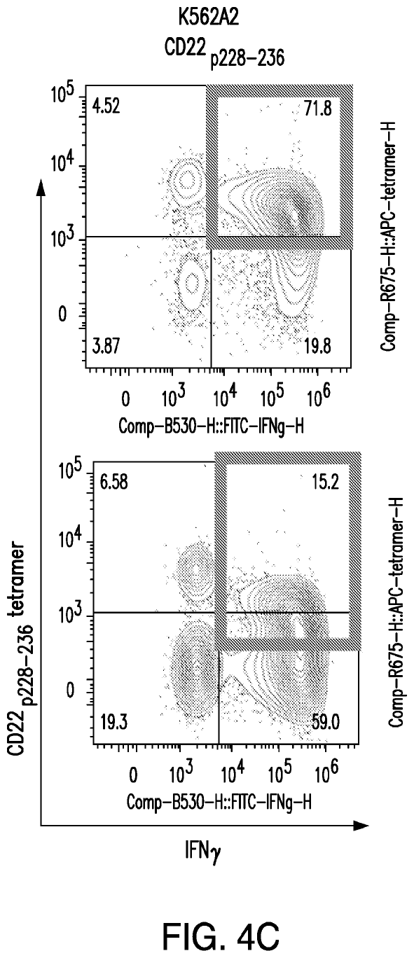

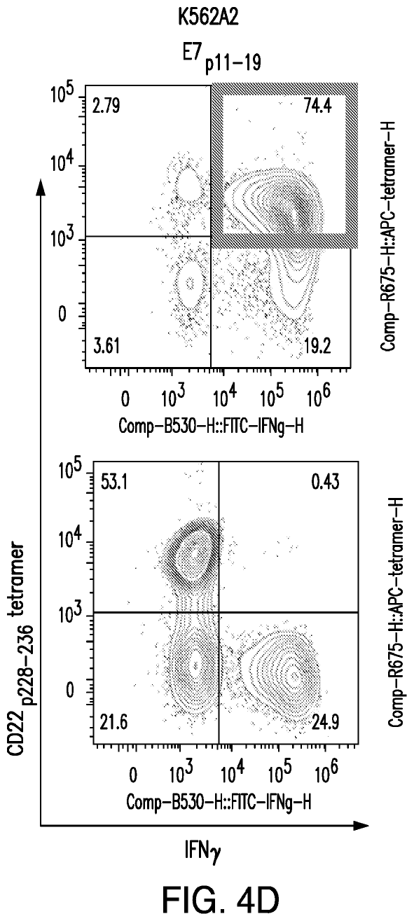

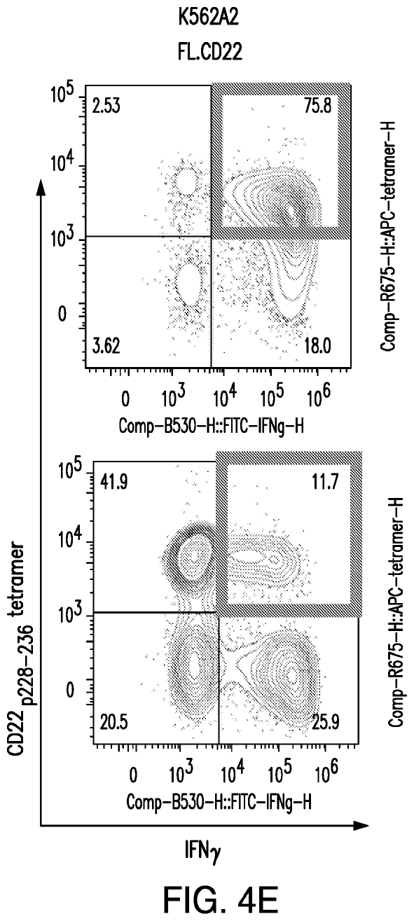

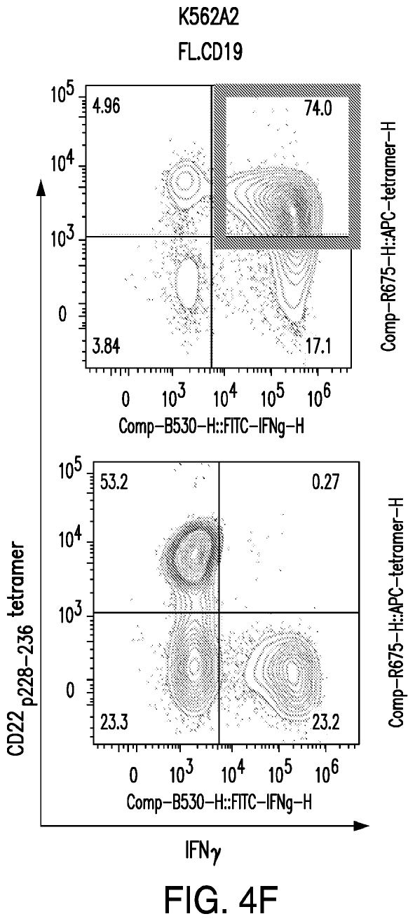

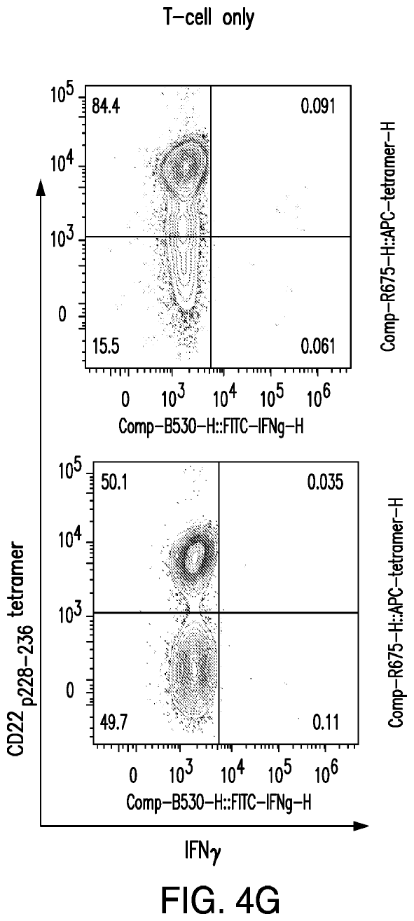

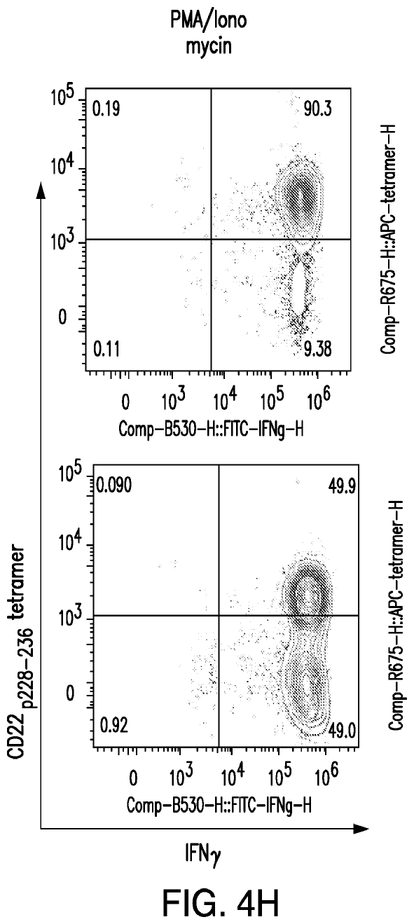

Specificity of tetramer+ population was assessed by co-staining of p-MHC tetramer and intracellular IFNγ. T cells were cultured with each target cell line at E:T=1:1 ratio. One hour after starting the co-culture, monensin/Brefeldin A was added. Following a total of 4 hours of co-culture, cells were first stained with live/dead discrimination dye, cell surface staining including p-MHC tetramers, then finally stained for intracellular IFNγ. Events were acquired and analyzed with flow cytometry (BD LSRFortessa). See FIGS. 4A-4H, each of which present a TCR example that was non-specific (top) and a TCR example that was specific (D146W8 at bottom).

The co-culture well denoted as D146W8 contained a T cell population that binds to the p-MHC tetramer and produces IFNγ in response to K562A2 CD22 p228-236 and K562A2-CD22 (full length CD22, F.L.CD22; highlighted with squares in FIGS. 4C and 4E). The non-specific TCR produced IFNγ non-specifically in recognition of HLA-A2+ cell line even without the target protein CD22 expression (highlighted with squares in FIGS. 4B-4F). From D146W8, tetramer-bound population was isolated using magnetic isolation.

Paired single cell TCR sequencing was performed to identify paired T-cell receptor (TCR) TCRα and TCRβ chain sequences (TCRα (TCRAV) and TCRβ (TCRBV)) (10× Genomics, Pleasanton, CA, USA). Sequencing was performed on bulk lymphocytes and/or T cells enriched for tetramer-bound populations as described above. One predominant clonotype was found (95.5% of the sequenced T cell population), which was named TCR clone D146W8 (named after the well ID from which the TCR was isolated).

### Example 2

This Example demonstrates construction of α retroviral vector encoding an anti-CD22 TCR, in accordance with aspects of the invention.

An MSGV1 based-retroviral vector was constructed which encoded the TCR alpha and beta chain variable regions of the TCR of Example 1 with the exception that the amino acid residue at position 2 of the N-terminal signal peptide of the beta chain was changed to an alanine in order to facilitate cloning into the vector. Additional modifications to the wild-type TCR were made, as described in more detail below.

Construction of the CD22-specific TCR was done as previously described (Jin et al., JCI Insight, 3(8): e99488 (2018), incorporated herein by reference in its entirety). Briefly, the TCRβ VDJ regions were fused to the mouse TCRβ constant chain, and the TCRα VJ regions were fused to the mouse TCRα constant chain. Without being bound to a particular theory or mechanism, it is believed that using the murine constant regions improves TCR expression and functionality (Cohen et al., Cancer Res., 66(17): 8878-8886 (2006)).

In addition, the murine TCRα and TCRβ constant chains were cysteine-modified, and transmembrane hydrophobic mutations were introduced into the murine TCRα constant chain. Without being bound to a particular theory or mechanism, it is believed that these modifications result in preferential pairing of the introduced TCβ chains and enhanced TCR surface expression and functionality (Cohen et al., Cancer Res., 67(8):3898-903 (2007); Haga-Friedman et al., J. Immu., 188: 5538-5546 (2012)).

The TCRβ and TCRα chains were separated by a Furin SGSG P2A linker (RAKRSGSGATNFSLLKQAGDVEENPGP) (SEQ ID NO: 27) to ensure a comparable expression efficiency of the two chains (Szymczak et al., Nat. Biotechnol., 22(5):589-94 (2004)).

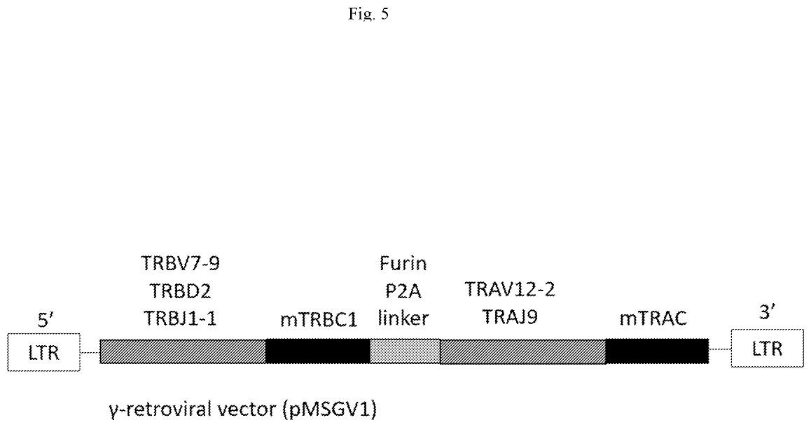

To allow cloning of the CD22-specific TCR expression cassette into the MSGV1 vector 5′NcoI site, the second amino acid in the TCRVP chain (the second amino acid within the N-terminal signal peptide) was changed to an alanine (A). The expression cassette had the following configuration: 5′NcoI-VDJβ-mCβ-Furin/SGSG/P2A-VJα-mCα-SalI3′. The nucleotide sequences of each TCR were codon optimized for human tissue expression. See FIG. 5. This example describes a synthesis of bicistronic vector in 5′TCRβ to TCRα 3′ orientation, but the order of TCRβ to TCRα can be reversed. The vector insert sequences were codon optimized for an expression in human tissues.

The modifications described in this Example were made, and the TCR variable regions had sequences as in the following table. Signal sequences were predicted using the IMGT database.

TABLE 7

| TCR chain | Amino acid sequence |
|---|---|
| Alpha variable | MKSLRVLLVILWLQLSWVWSQQKEVEQNSGPLSV |
| (TRAV12-2_TRAJ9) | PEGAIASLNCTYSDRGSQSFFWYRQYSGKSPELI |
| (with N-terminal | MFIYSNGDKEDGRFTAQLNKASQYVSLLIRDSQP |
| signal peptide) | SDSATYLCAVKGAGGFKTIFGAGTRLFVKA |
| | (SEQ ID NO: 7) |
| Beta variable | MATSLLCWMALCLLGADHADTGVSQNPRHKITKR |
| (TRBV7- | GQNVTFRCDPISEHNRLYWYRQTLGQGPEFLTYF |
| 9 TRBD2_TRBJ1-1) | QNEAQLEKSRLLSDRFSAERPKGSFSTLEIQRTE |
| (with N-terminal | QGDSAMYLCASSPGNTEAFFGQGTRLTVV (SEQ |
| signal peptide) | ID NO: 9) |
| Alpha variable | QKEVEQNSGPLSVPEGAIASLNCTYSDRGSQSFF |
| (TRAV12-2_TRAJ9) | WYRQYSGKSPELIMFIYSNGDKEDGRFTAQLNKA |
| (without | SQYVSLLIRDSQPSDSATYLCAVKGAGGFKTIFG |
| N-terminal | AGTRLFVKA (SEQ ID NO: 10) |
| signal peptide) | |
| Beta variable | DTGVSQNPRHKITKRGQNVTFRCDPISEHNRLYW |
| (TRBV7- | YRQTLGQGPEFLTYFQNEAQLEKSRLLSDRFSAE |
| 9 TRBD2_TRBJ1-1) | RPKGSFSTLEIQRTEQGDSAMYLCASSPGNTEAF |
| (without | FGQGTRLTVV (SEQ ID NO: 11) |
| N-terminal | |
| signal peptide) | |

### Example 3

This Example demonstrates characterization of the TCR of Example 2, in accordance with aspects of the invention.

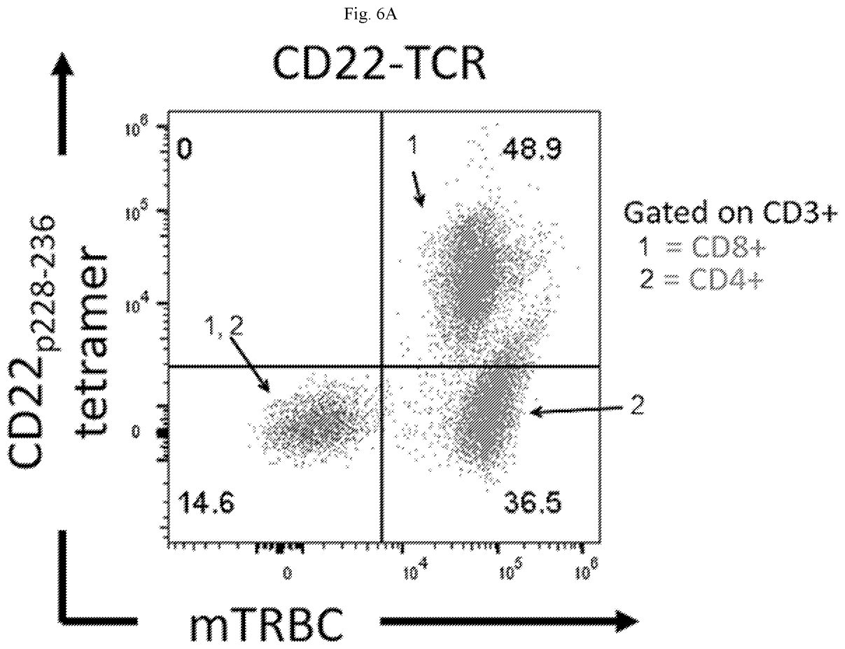

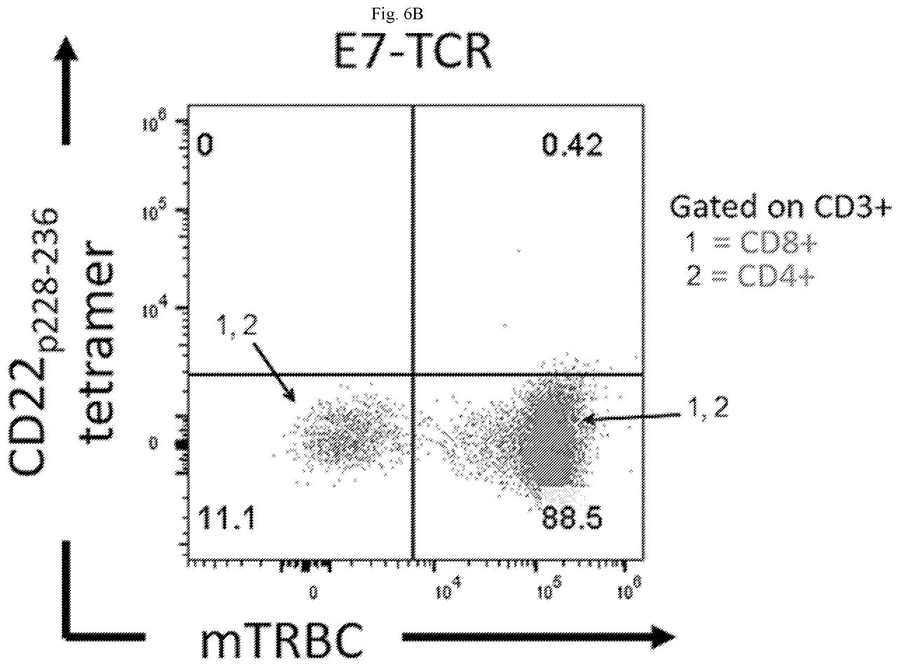

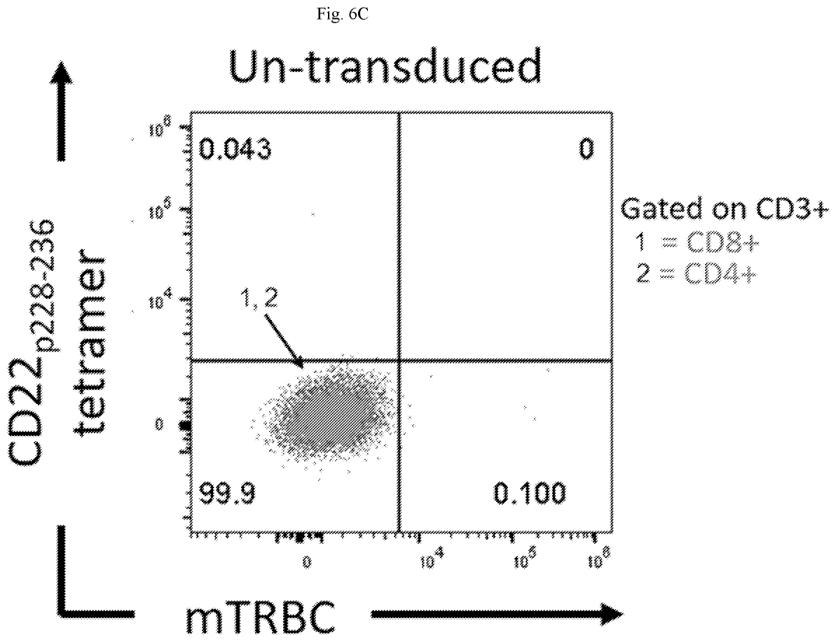

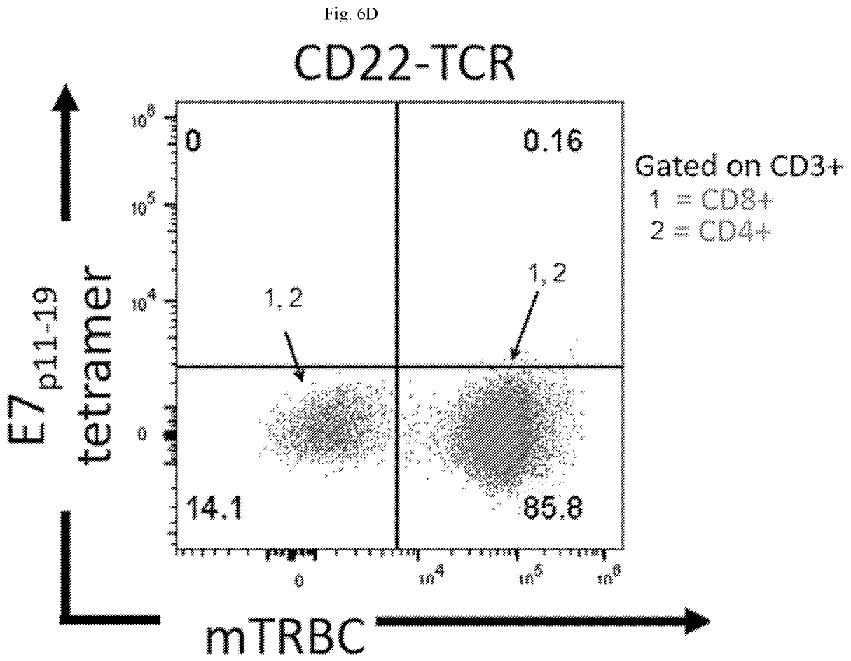

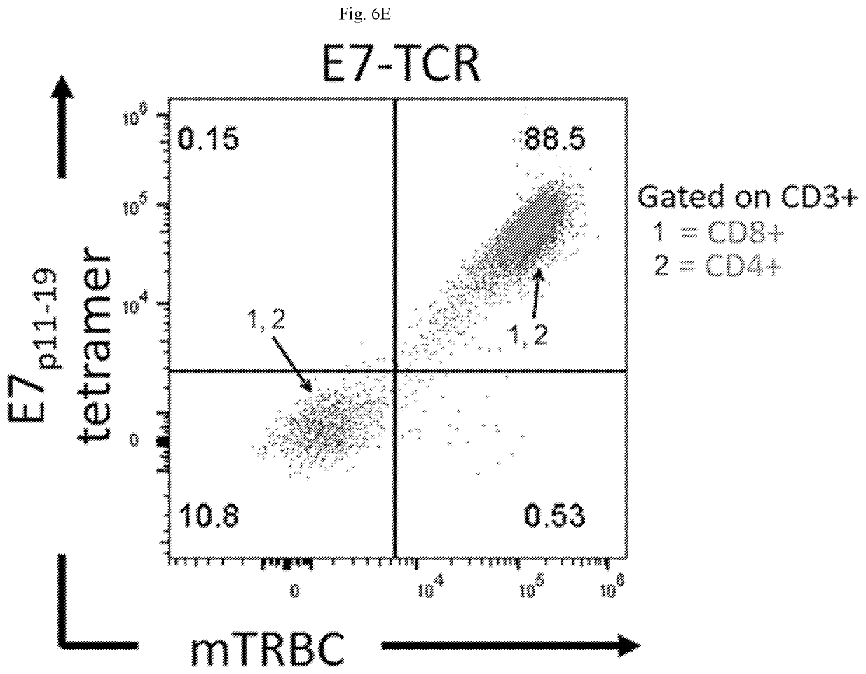

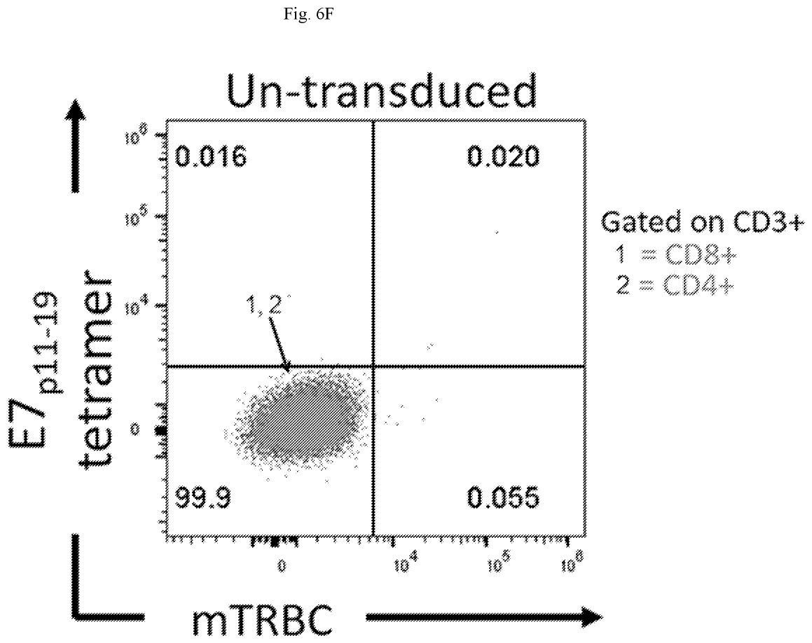

Gamma retroviral supernatant was produced by transfecting 293GP cells with D146W8 vector plasmid of Example 2 and with RD114 envelope plasmid. FIGS. 6A-6F represent T cells transduced with supernatant collected 48 hours post transfection. Transduction was performed on a total of 4 different PBMC donors on two independent occasions (2 donors each time). FACS was run 6 days after the completion of transduction (8 days after T-cell activation with OKT3/IL-2). Similar CD22 p228-236 tetramer staining pattern was observed regardless of PBMC donor's HLA-A2 status. (UV exchange tetramer production (BioLegend Flex-T HLA-A*02:01 monomer); stain with antibodies/tetramer at 2-8° C. at dark for 30 min, followed by FACS gated on singlet/live/lymphocytes).

### Example 4

This Example demonstrates that the inventive TCR has high functional avidity, in accordance with aspects of the invention.

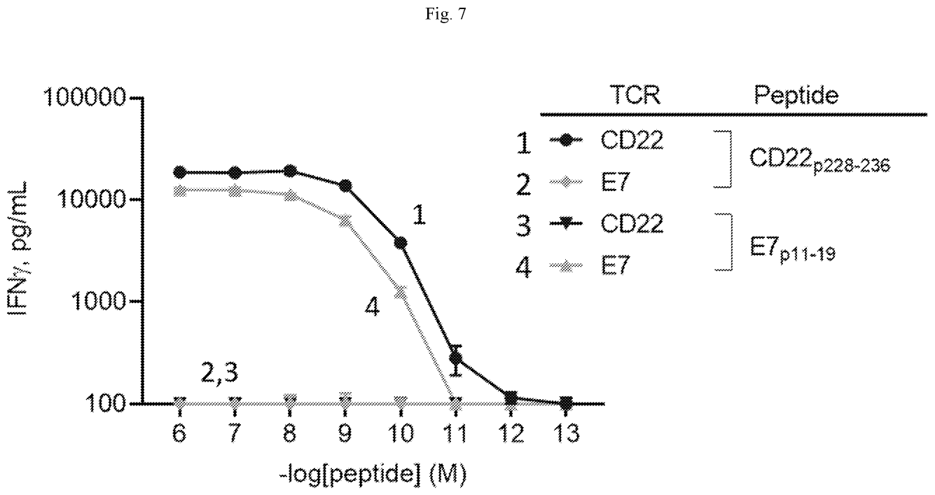

T cells transduced with the anti-CD22 TCR of Example 2 or an anti-E7 TCR were co-cultured with an HLA-A2+ cell line (K562A2) loaded with different concentrations of cognate peptides at an E:T ratio of 1:1 (5e4 cells each in a total volume of 200 uL/well in 96-well U-bottom plate). Overnight co-culture supernatant was obtained and IFNγ levels were measured with ELISA. FIG. 7 presents the results.

### Example 5

This Example demonstrates that T cells engineered to express the inventive TCR recognized CD22+/HLA-A2+ cell lines, in accordance with aspects of the invention.

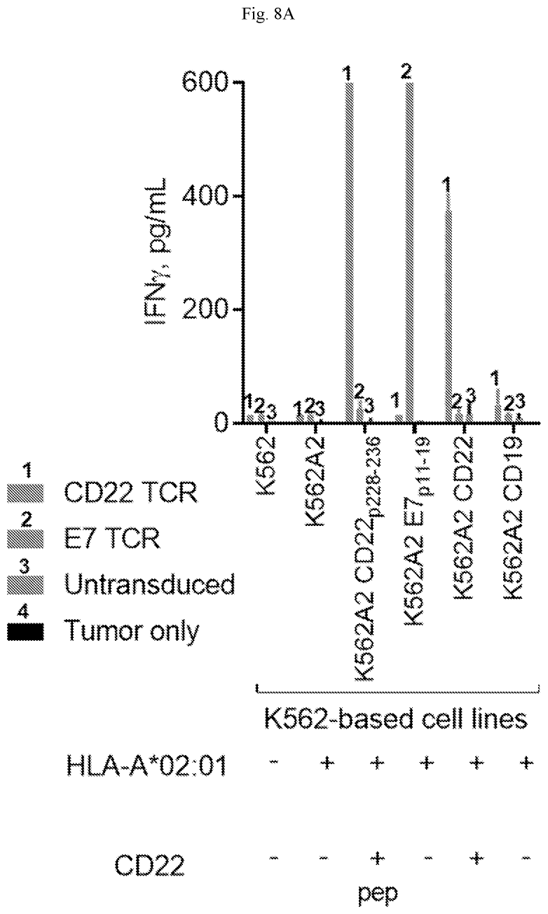

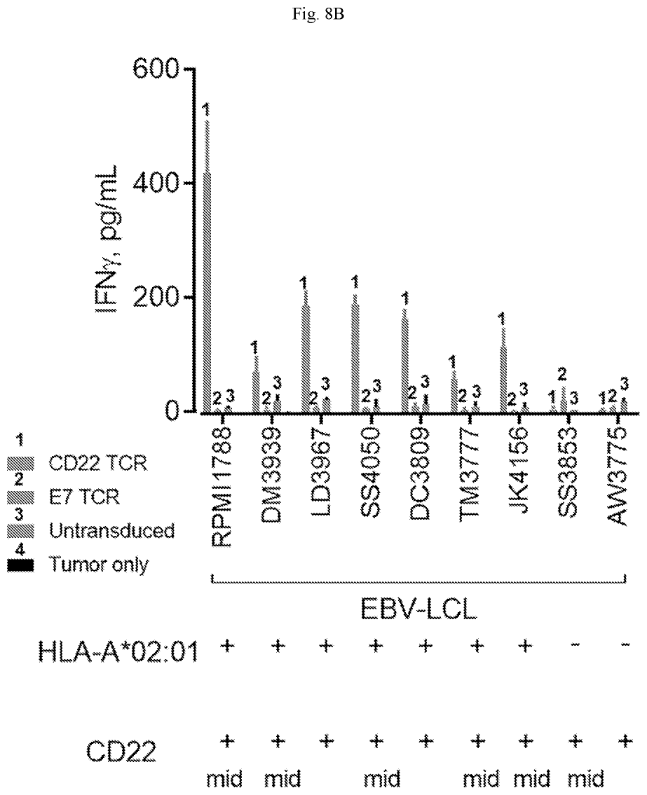

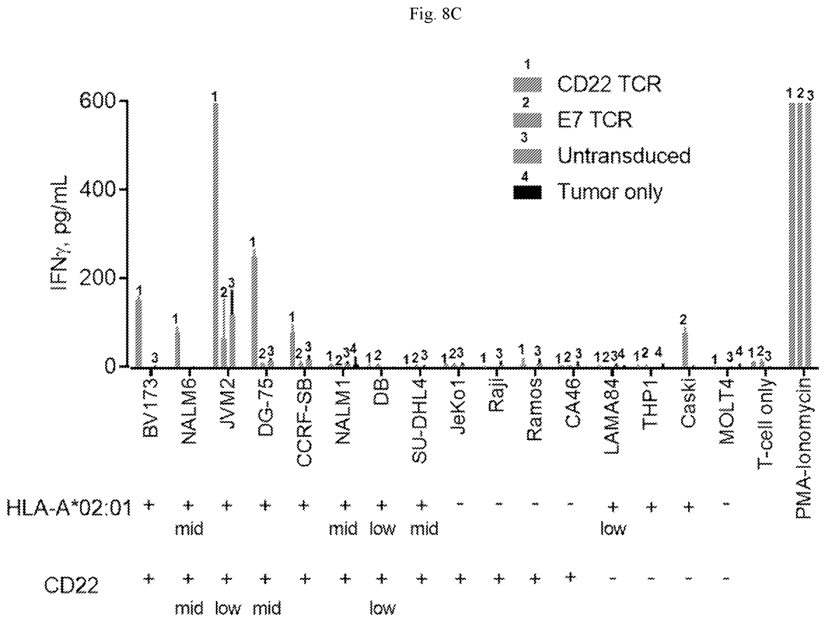

T cells transduced with the anti-CD22 TCR of Example 2 or an anti-E7 TCR were co-cultured with each of the target cell lines shown in FIGS. 8A-8C at an E:T ratio of 1:1 (5e4 cells each in a total volume of 200 μL/well in 96-well U-bottom plate). Overnight co-culture supernatant was obtained and IFNγ levels were measured with ELISA. FIGS. 8A-8C present the results. The target cell lines included: NALM6, CCRF-SB, Raji, Ramos, DG75, CA46, NALM1, BV173, SU-DHL-4, DB, Jeko-1, RPM11788, MOLT4, CaSki.

### Example 6

This Example demonstrates that the inventive TCR-transduced T cells are polyfunctional.

T cells transduced with the TCR of Example 2 were cultured with each target cell lines at E:T=1:1 ratio. One hour after starting the co-culture, monensin/Brefeldin A was added. Following a total of 4 hours of co-culture, cells were first stained with live/dead discrimination dye, cell surface staining including p-MHC tetramers, then finally stained for intracellular IFNγ, TNFα, and IL-2. Events were acquired and analyzed with flow cytometry (BD LSRFortessa).

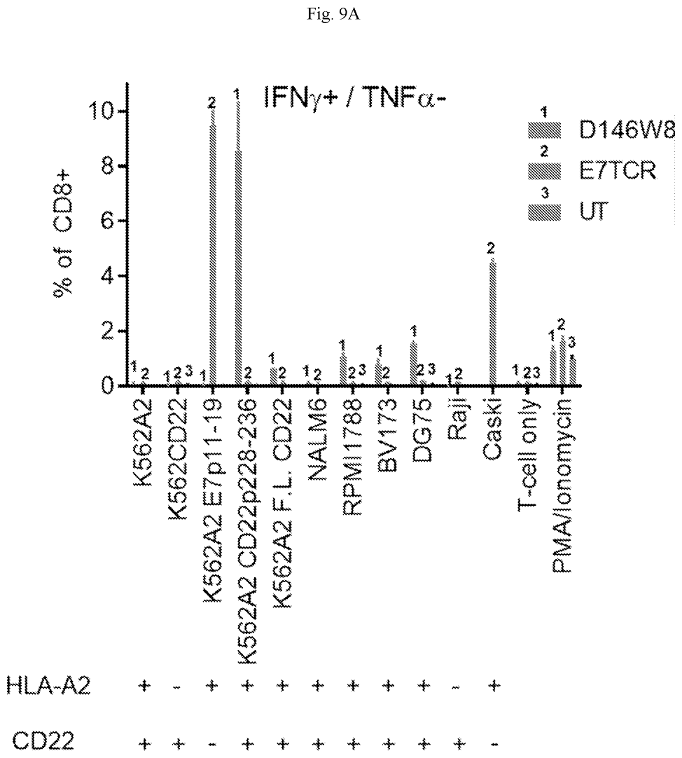

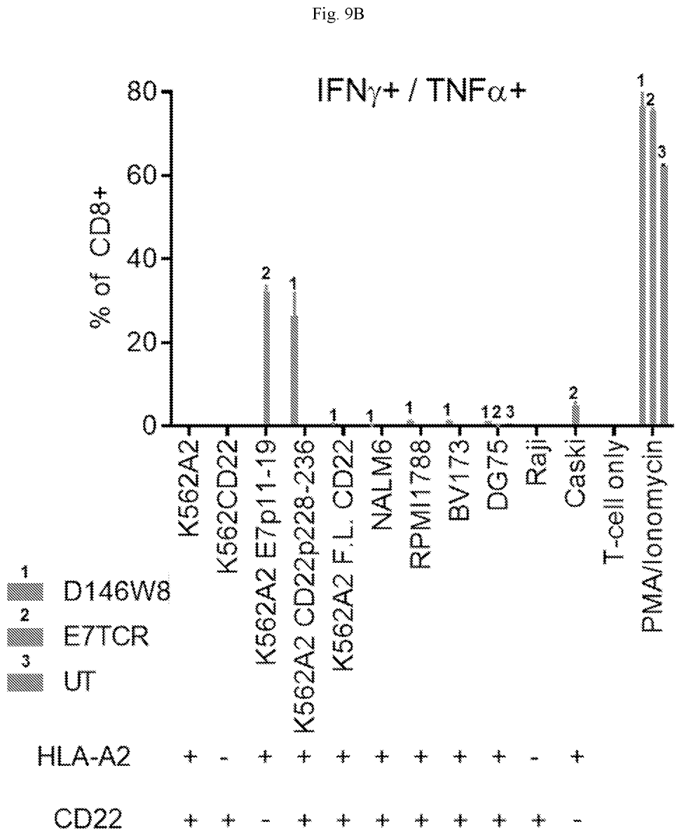

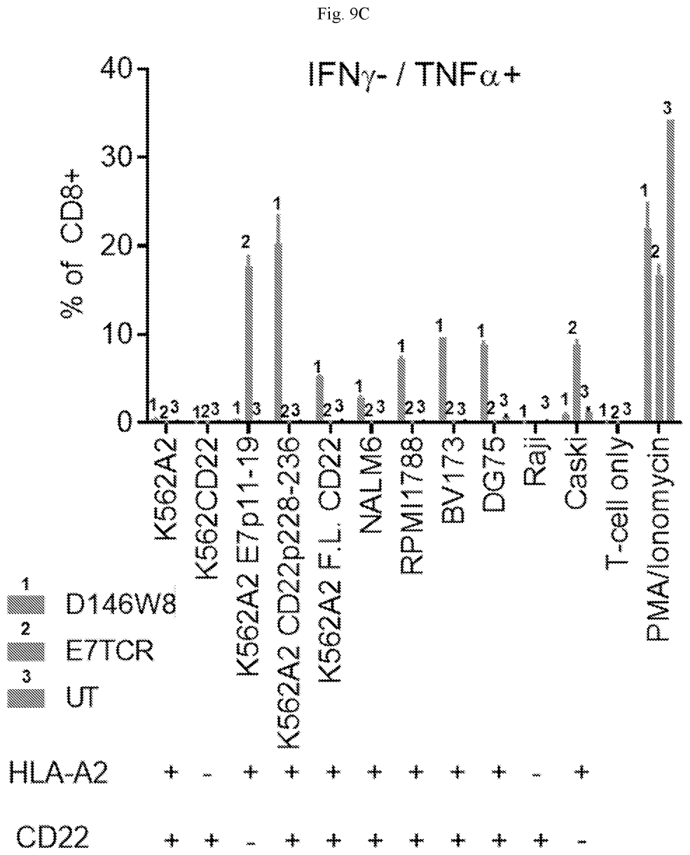

The frequencies of populations that are IFNγ+/TNFα−, IFNγ+/TNFα+, and IFNγ−/TNFα+ are shown in FIGS. 9A, 9B, and 9C, respectively.

### Example 7

This Example demonstrates that CD22 p228-236 residues involved in MHC binding and recognition by the inventive TCR.

T cells transduced with the anti-CD22 TCR of Example 2 or an anti-E7 TCR were co-cultured with an HLA-A2+ cell line (K562A2) loaded with 1 μM of peptide as shown in the table below.

TABLE 8

| | | | | Predicted | |
|---|---|---|---|---|---|
| | | | Predicted | HLA- | |
| Alanine | | SEQ | HLA- | A*02:01 | TCR |
| substi- | | ID | A*02:01 | anchor | recog- |
| tution | Peptide | NO. | IC₅₀ | residue | nition |
| WT | FLSNDTVQL | 12 | 5.8 | | |
| 1. F > A | ALSNDTVQL | 35 | 80.7 | X | |
| 2. L > A | FASNDTVQL | 36 | 1974.1 | X | |
| 3. S > A | FLANDTVQL | 37 | 4.4 | | X |
| 4. N > A | FLSADTVQL | 38 | 5.1 | | X |
| 5. D > A | FLSNATVQL | 39 | 5.3 | | X |
| 6. T > A | FLSNDAVQL | 40 | 10.2 | | |
| 7. V > A | FLSNDTAQL | 41 | 6.8 | | |
| 8. Q > A | FLSNDTVAL | 42 | 5.5 | | X |
| 9 L > A | FLSNDTVQA | 43 | 26.8 | X | |

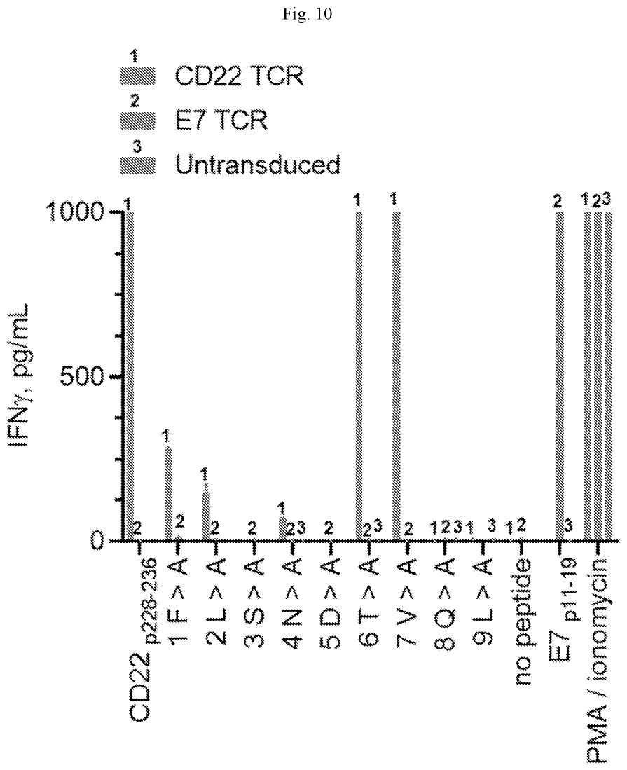

Alanine scanning was performed to determine which residues were involved in binding and recognition. Overnight co-culture supernatant was obtained and IFNγ levels were measured with ELISA. FIG. 10 shows the results. IC50 prediction was performed using the IEDB analysis resource Consensus tool (Kim et al., Nucleic Acids Res., 40: W525-30 (2012), incorporated by reference herein) which combines predictions from ANN aka NetMHC (Nielsen et al., Protein Sci., 12: 1007-1017 (2003); Lundegaard et al., Nucleic Acids Res., 36: W509-512 (2008); Andreatta et al., Bioinformatics, 32:511-7 (2016), incorporated by reference herein), SMM (Peters et al., BMC Bioinformatics, 6: 132 (2005), incorporated by reference herein) and Comblib (Sidney et al., Immunome Res., 4:2 (2008), incorporated by reference herein).

Substitution of the first, second and 9th residues to alanine resulted in an inhibition of IFNγ production and increase in predicted HLA-A*02:01 IC₅₀, implying that these residues are HLA-A*02:01 anchor residues. Substitution of the third, fourth, fifth, and eighth residues to alanine resulted in an inhibition of IFNγ production despite HLA-A*02:01 IC₅₀ remaining the same, and thus these are the residues involved in the TCR recognition.

### Example 8

This Example demonstrates that the inventive TCR has no cross reactivity against candidate peptides tested.

Based on the TCR recognition motif determined in the alanine substitution assay of Example 7, an in silico search was performed for human peptides with amino acid sequences that are similar to the TCR recognition motif and/or human peptides with high levels of sequence homology (ScanProsite tool: peptides with amino acid sequences that have the same TCR recognition motif or have substitutions of residues with biochemically similar amino acids (De Castro et al., Nucleic Acids Res., 34(Web Server issue): W362-5 (2006)); NCBI protein BLAST: peptides with high level of sequence homology in human proteome).

T cells transduced with the anti-CD22 TCR of Example 2 were co-cultured with K562A2 loaded with 1 μM of each of the candidate peptides at an E:T ratio of 1:1 (5e4 cells each in a total volume of 200 μL/well in 96-well U-bottom plate). Overnight co-culture supernatant was obtained and IFNγ levels were measured with ELISA.

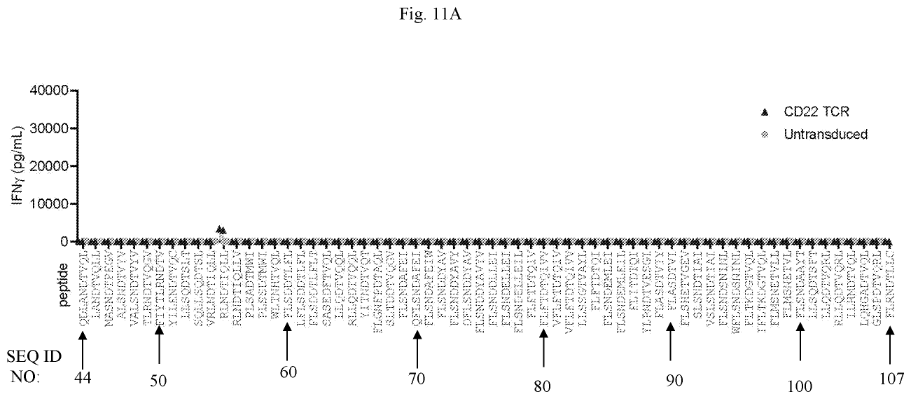

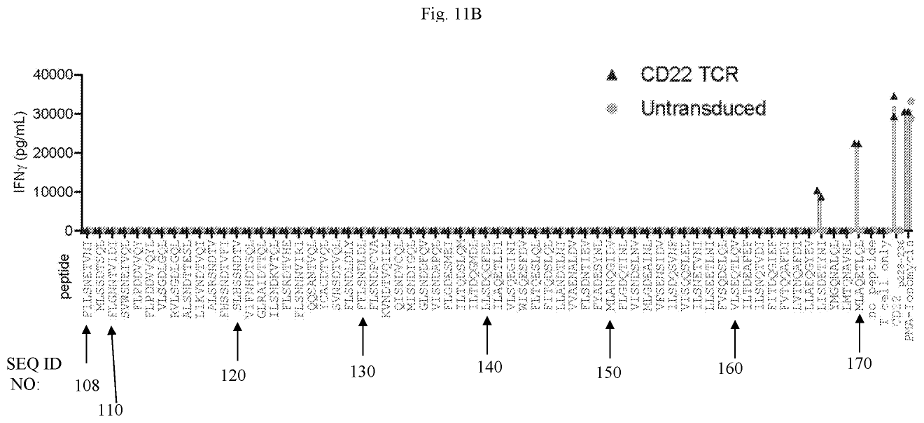

Of the candidate peptides tested, T cells transduced with the anti-CD22 TCR produced low levels of IFNγ in recognition of K562A2 cells loaded with supraphysiological concentration (1 μM) of peptides RLNDTIQLL (SEQ ID NO: 55; derived from protein UHRF1), LISDETYNI (SEQ ID NO: 167; derived from protein TMM68 (or TMEM68)), and MLAQETLQL (SEQ ID NO: 170; derived from protein WHAL1 (or WHAMMP3)) (FIGS. 11A, 11B).

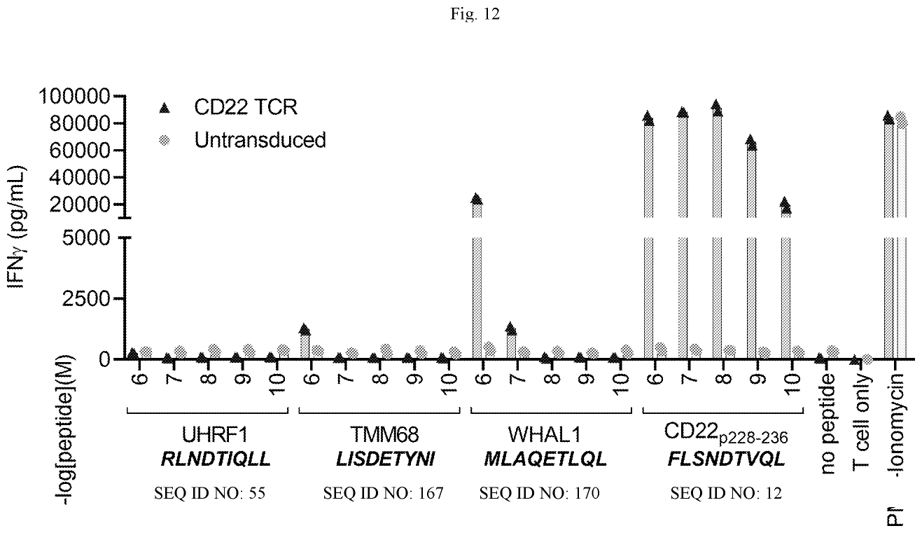

The peptides that demonstrated potential cross-reactivity at supraphysiological concentrations (FIGS. 11A and 11B) were next evaluated as to whether they activate T cells at physiologically relevant concentrations. T cells transduced with the anti-CD22 TCR were co-cultured with K562A2 loaded with titrated concentrations of peptides as indicated in the x-axis of FIGS. 11A and 11B. Overnight co-culture supernatant was harvested and the levels of IFNγ was evaluated with ELISA. The anti-CD22 TCR recognized only the intended CD22 p228-236 epitope at physiologically relevant concentrations (FIG. 12).

### Example 9

This Example demonstrates that the inventive TCR is effective in vivo in reducing tumor burden.

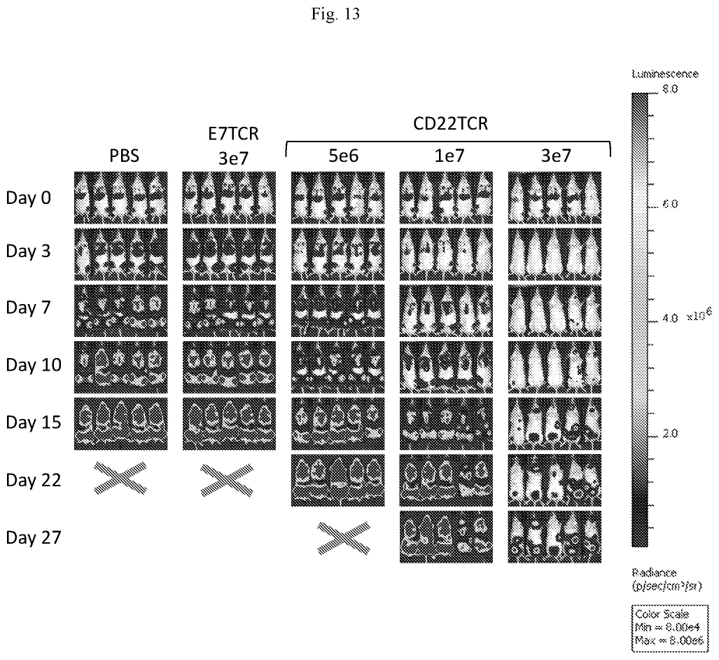

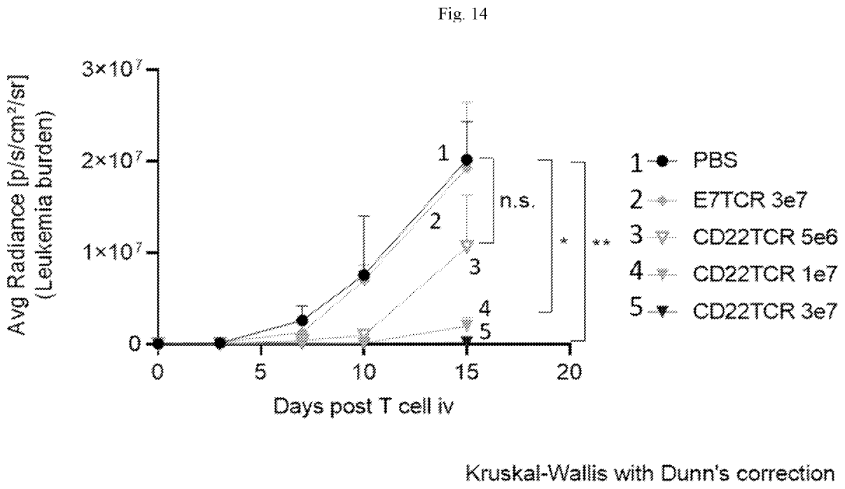

A leukemia cell line BV173 (CML blast crisis, B-ALL transformation) was engineered to express fire-fly luciferase and GFP (BV173-ffLuc-GFP). NSG mice (NOD-scid IL2Rgamma^{null}) were injected with leukemia cells (1e6) via tail vein. Seven days after leukemia injection, T cells transduced to express either the anti-CD22 TCR or the irrelevant E7 TCR were injected via tail vein. E7 TCR-transduced T cells were given 3e7 cells/mouse, and anti-CD22 TCR-transduced T cells were given 5e6 cells/mouse, 1e7 cells/mouse, or 3e7 cells/mouse as indicated in the FIG. 13. Bioluminescence images were obtained using the IVIS Spectrum in vivo imaging system (PerkinElmer) immediately before T cell injection and every 3-7 days after T cell injection to evaluate chronological changes in leukemia burden. FIG. 13 shows the IVIS images (scale is set to the radiance of 8e4-8e6 p/sec/cm2/sr, and gray/black areas in mouse body indicate the presence of leukemia). FIG. 14 is a plot of average radiance (p/s/cm2/sr), which is a proxy for the systemic leukemia burden of each mouse. Statistical analysis comparing groups of interest was performed using Kruskal-Wallis with Dunn's correction.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the present disclosure (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, and/or exemplary language (e.g., “such as”), does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated or purified T-cell receptor (TCR), wherein the TCR has antigenic specificity for a CD22 amino acid sequence presented by a human leukocyte antigen (HLA) Class I molecule, the TCR comprising the amino acid sequences of
(a) SEQ ID NOs: 1-3,
(b) SEQ ID NOs: 4-6, or
(c) SEQ ID NOs: 1-6.

2. The isolated or purified TCR according to claim 1, wherein the CD22 amino acid sequence is from human CD22.

3. The isolated or purified TCR according to claim 1, wherein the CD22 amino acid sequence is SEQ ID NO: 12.

4. The isolated or purified TCR according to claim 1, wherein the HLA Class I molecule is an HLA-A molecule.

5. The isolated or purified TCR according to claim 1, wherein the HLA Class I molecule is an HLA-A*02 molecule.

6. The isolated or purified TCR according to claim 1, wherein the HLA Class I molecule is encoded by the HLA-A*02:01 allele.

7. The isolated or purified TCR according to claim 1, comprising the amino acid sequence(s) of:
(i) SEQ ID NO: 7;
(ii) SEQ ID NO: 8 or 9;
(iii) SEQ ID NO: 10;
(iv) SEQ ID NO: 11;
(v) both of SEQ ID NO: 7, and SEQ ID NO: 8 or 9; or
(vi) both of SEQ ID NO: 10 and SEQ ID NO: 11.

8. The isolated or purified TCR of claim 1, further comprising:
(a) an α chain constant region comprising the amino acid sequence of SEQ ID NO: 20, wherein:
(i) X at position 48 of SEQ ID NO: 20 is Thr or Cys;
(ii) X at position 112 of SEQ ID NO: 20 is Ser, Ala, Val, Leu, Ile, Pro, Phe, Met, or Trp;
(iii) X at position 114 of SEQ ID NO: 20 is Met, Ala, Val, Leu, Ile, Pro, Phe, or Trp; and
(iv) X at position 115 of SEQ ID NO: 20 is Gly, Ala, Val, Leu, Ile, Pro, Phe, Met, or Trp;
(b) a β chain constant region comprising the amino acid sequence of SEQ ID NO: 21, wherein X at position 57 of SEQ ID NO: 21 is Ser or Cys; or
(c) both (a) and (b).

9. The isolated or purified TCR of claim 1, comprising:
(a) an α chain comprising the amino acid sequence of SEQ ID NO: 22, wherein: (i) X at position 180 of SEQ ID NO: 22 is Thr or Cys; (ii) X at position 244 of SEQ ID NO: 22 is Ser, Ala, Val, Leu, Ile, Pro, Phe, Met, or Trp; (iii) X at position 246 of SEQ ID NO: 22 is Met, Ala, Val, Leu, Ile, Pro, Phe, or Trp; and (iv) X at position 247 of SEQ ID NO: 22 is Gly, Ala, Val, Leu, Ile, Pro, Phe, Met, or Trp;
(b) a β chain comprising the amino acid sequence of SEQ ID NO: 23 or 24, wherein X at position 188 of SEQ ID NO: 23 or 24 is Ser or Cys;
(c) both (a) and (b);
(d) an α chain comprising the amino acid sequence of SEQ ID NO: 25, wherein: (i) X at position 159 of SEQ ID NO: 25 is Thr or Cys; (ii) X at position 223 of SEQ ID NO: 25 is Ser, Ala, Val, Leu, Ile, Pro, Phe, Met, or Trp; (iii) X at position 225 of SEQ ID NO: 25 is Met, Ala, Val, Leu, Ile, Pro, Phe, or Trp; and (iv) X at position 226 of SEQ ID NO: 25 is Gly, Ala, Val, Leu, Ile, Pro, Phe, Met, or Trp;
(e) a β chain comprising the amino acid sequence of SEQ ID NO: 26, wherein X at position 169 of SEQ ID NO: 26 is Ser or Cys; or
(f) both (d) and (e).

10. An isolated or purified polypeptide comprising a functional portion of the TCR of claim 1, wherein the functional portion comprises the amino acid sequences of:
(a) SEQ ID NOs: 1-3,
(b) SEQ ID NOs: 4-6, or
(c) SEQ ID NOs: 1-6.

11. The isolated or purified polypeptide according to claim 10, wherein the functional portion comprises the amino acid sequence(s) of:
(i) SEQ ID NO: 7;
(ii) SEQ ID NO: 8 or 9;
(iii) SEQ ID NO: 10;
(iv) SEQ ID NO: 11;
(v) both of SEQ ID NO: 7, and SEQ ID NO: 8 or 9; or
(vi) both of SEQ ID NO: 10 and SEQ ID NO: 11.

12. The isolated or purified polypeptide of claim 10, further comprising:
(a) the amino acid sequence of SEQ ID NO: 20, wherein:
(i) X at position 48 of SEQ ID NO: 20 is Thr or Cys;
(ii) X at position 112 of SEQ ID NO: 20 is Ser, Ala, Val, Leu, Ile, Pro, Phe, Met, or Trp;
(iii) X at position 114 of SEQ ID NO: 20 is Met, Ala, Val, Leu, Ile, Pro, Phe, or Trp; and
(iv) X at position 115 of SEQ ID NO: 20 is Gly, Ala, Val, Leu, Ile, Pro, Phe, Met, or Trp;
(b) the amino acid sequence of SEQ ID NO: 21, wherein X at position 57 of SEQ ID NO: 21 is Ser or Cys; or
(c) both (a) and (b).

13. The isolated or purified polypeptide of claim 10, comprising:
(a) the amino acid sequence of SEQ ID NO: 22, wherein: (i) X at position 180 of SEQ ID NO: 22 is Thr or Cys; (ii) X at position 244 of SEQ ID NO: 22 is Ser, Ala, Val, Leu, Ile, Pro, Phe, Met, or Trp; (iii) X at position 246 of SEQ ID NO: 22 is Met, Ala, Val, Leu, Ile, Pro, Phe, or Trp; and (iv) X at position 247 of SEQ ID NO: 22 is Gly, Ala, Val, Leu, Ile, Pro, Phe, Met, or Trp;
(b) the amino acid sequence of SEQ ID NO: 23 or 24, wherein X at position 188 of SEQ ID NO: 23 or 24 is Ser or Cys;
(c) both (a) and (b);
(d) the amino acid sequence of SEQ ID NO: 25, wherein: (i) X at position 159 of SEQ ID NO: 25 is Thr or Cys; (ii) X at position 223 of SEQ ID NO: 25 is Ser, Ala, Val, Leu, Ile, Pro, Phe, Met, or Trp; (iii) X at position 225 of SEQ ID NO: 25 is Met, Ala, Val, Leu, Ile, Pro, Phe, or Trp; and (iv) X at position 226 of SEQ ID NO: 25 is Gly, Ala, Val, Leu, Ile, Pro, Phe, Met, or Trp;
(e) the amino acid sequence of SEQ ID NO: 26, wherein X at position 169 of SEQ ID NO: 26 is Ser or Cys;
(f) both (d) and (e).

14. (canceled)

15. The isolated or purified polypeptide according to claim 10, comprising:
a first polypeptide chain comprising the amino acid sequences of SEQ ID NOs: 1-3 and a second polypeptide chain comprising the amino acid sequences of SEQ ID NOs: 4-6.

16. The isolated or purified polypeptide according to claim 10, comprising:
(i) a first polypeptide chain comprising the amino acid sequence of SEQ ID NO: 7 and a second polypeptide chain comprising the amino acid sequence of SEQ ID NO: 8 or 9; or
(ii) a first polypeptide chain comprising the amino acid sequence of SEQ ID NO: 10 and a second polypeptide chain comprising the amino acid sequence of SEQ ID NO: 11.

17. The isolated or purified polypeptide of claim 10, further comprising:
(a) a first polypeptide chain comprising the amino acid sequence of SEQ ID NO: 20, wherein:
(i) X at position 48 of SEQ ID NO: 20 is Thr or Cys;
(ii) X at position 112 of SEQ ID NO: 20 is Ser, Ala, Val, Leu, Ile, Pro, Phe, Met, or Trp;
(iii) X at position 114 of SEQ ID NO: 20 is Met, Ala, Val, Leu, Ile, Pro, Phe, or Trp; and
(iv) X at position 115 of SEQ ID NO: 20 is Gly, Ala, Val, Leu, Ile, Pro, Phe, Met, or Trp;
(b) a second polypeptide chain constant region comprising the amino acid sequence of SEQ ID NO: 21, wherein X at position 57 of SEQ ID NO: 21 is Ser or Cys; or
(c) both (a) and (b).

18. The isolated or purified polypeptide of claim 10, comprising:
(a) a first polypeptide chain comprising the amino acid sequence of SEQ ID NO: 22, wherein: (i) X at position 180 of SEQ ID NO: 22 is Thr or Cys; (ii) X at position 244 of SEQ ID NO: 22 is Ser, Ala, Val, Leu, Ile, Pro, Phe, Met, or Trp; (iii) X at position 246 of SEQ ID NO: 22 is Met, Ala, Val, Leu, Ile, Pro, Phe, or Trp; and (iv) X at position 247 of SEQ ID NO: 22 is Gly, Ala, Val, Leu, Ile, Pro, Phe, Met, or Trp;
(b) a second polypeptide chain comprising the amino acid sequence of SEQ ID NO: 23 or 24, wherein X at position 188 of SEQ ID NO: 23 or 24 is Ser or Cys;
(c) both (a) and (b);
(d) a first polypeptide chain comprising the amino acid sequence of SEQ ID NO: 25, wherein: (i) X at position 159 of SEQ ID NO: 25 is Thr or Cys; (ii) X at position 223 of SEQ ID NO: 25 is Ser, Ala, Val, Leu, Ile, Pro, Phe, Met, or Trp; (iii) X at position 225 of SEQ ID NO: 25 is Met, Ala, Val, Leu, Ile, Pro, Phe, or Trp; and (iv) X at position 226 of SEQ ID NO: 25 is Gly, Ala, Val, Leu, Ile, Pro, Phe, Met, or Trp;
(e) a second polypeptide chain comprising the amino acid sequence of SEQ ID NO: 26, wherein X at position 169 of SEQ ID NO: 26 is Ser or Cys;
(f) both (d) and (e).

19. An isolated or purified nucleic acid comprising a nucleotide sequence encoding the TCR according to claim 1.

20. An isolated or purified nucleic acid comprising, from 5′ to 3′, a first nucleic acid sequence and a second nucleotide sequence, wherein the first and second nucleotide sequence, respectively, encode the amino sequences of SEQ ID NOs: 7, and 8 or 9; 8 or 9, and 7; 10 and 11; or 11 and 10.

21. The isolated or purified nucleic acid according to claim 20, further comprising a third nucleotide sequence interposed between the first and second nucleotide sequence, wherein the third nucleotide sequence encodes a cleavable linker peptide.

22. The isolated or purified nucleic acid according to claim 21, wherein the cleavable linker peptide comprises the amino acid sequence of SEQ ID NO: 27.

23. A recombinant expression vector comprising the nucleic acid according to claim 19.

24. The recombinant expression vector according to claim 23, which is a transposon or a lentiviral vector.

25. (canceled)

26. (canceled)

27. A method of producing a host cell expressing a TCR that has antigenic specificity for a CD22 amino acid sequence, the method comprising contacting a cell with the vector according to claim 23 under conditions that allow introduction of the vector into the cell.

28. An isolated or purified host cell comprising the nucleic acid according to claim 19.

29. The host cell according to claim 28 wherein the cell is a human lymphocyte.

30. The host cell according to claim 28, wherein the cell is selected from a T cell, a natural killer T (NKT) cell, an invariant natural killer T (iNKT) cell, and a natural killer (NK) cell.

31. An isolated or purified population of cells comprising the host cell according to claim 28.

32. A method of producing a TCR, polypeptide, or protein, the method comprising culturing the host cell according to claim 28 so that the TCR, polypeptide, or protein is produced.

33. A pharmaceutical composition comprising (a) the TCR according to claim 1, a nucleic acid encoding the TCR, a recombinant expression vector comprising the nucleic acid, a host cell comprising the expression vector or the nucleic acid, or a population of such cells and (b) a pharmaceutically acceptable carrier.

34. A method of detecting the presence of cancer in mammal, the method comprising:
(a) contacting a sample comprising cells of the cancer with the TCR according to claim 1, a nucleic acid encoding the TCR, a recombinant expression vector comprising the nucleic acid, a host cell comprising the expression vector or the nucleic acid, a population of such cells, or a pharmaceutical composition comprising any of these and a pharmaceutically acceptable carrier, thereby forming a complex; and
(b) detecting the complex,
wherein detection of the complex is indicative of the presence of cancer in the mammal.

35. A method for inducing an immune response against a cancer in a mammal or for treating or preventing cancer in a mammal, the method comprising administering the pharmaceutical composition of claim 33 to the mammal at a dose and manner of administration sufficient to induce an immune response against a cancer or treat or prevent cancer in the mammal.

36. (canceled)

37. The method according to claim 34, wherein the cancer expresses a CD22 amino acid sequence.

38. The method according to claim 34, wherein the cancer is Hodgkin lymphoma, T/NK cell lymphoma, post-transplant lymphoproliferative disorders, nasopharyngeal cancer, or gastric cancer.

39. The method according to claim 35, wherein the cancer expresses a CD22 amino acid sequence.

40. The method according to claim 35, wherein the cancer is Hodgkin lymphoma, T/NK cell lymphoma, post-transplant lymphoproliferative disorders, nasopharyngeal cancer, or gastric cancer.


SEQUENCE LISTING (170 sequences)

SEQ ID NO: 1 - protein, 6 aa, Homo sapiens
DRGSQS

SEQ ID NO: 2 - protein, 6 aa, Homo sapiens
IYSNGD

SEQ ID NO: 3 - protein, 11 aa, Homo sapiens
AVKGAGGFKTI

SEQ ID NO: 4 - protein, 5 aa, Homo sapiens
SEHNR

SEQ ID NO: 5 - protein, 6 aa, Homo sapiens
FQNEAQ

SEQ ID NO: 6 - protein, 10 aa, Homo sapiens
ASSPGNTEAF

SEQ ID NO: 7 - protein, 132 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKGAGGFKTIFGAGTRLFVKA

SEQ ID NO: 8 - protein, 131 aa, Homo sapiens
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSPGNTEAFFGQGTRLTVV

SEQ ID NO: 9 - protein, 131 aa, Artificial Sequence
  synthetic
MATSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSPGNTEAFFGQGTRLTVV

SEQ ID NO: 10 - protein, 111 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVKGAGGFKTIFGAGTRLFVKA

SEQ ID NO: 11 - protein, 112 aa, Homo sapiens
DTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSPGNTEAFFGQGTRLTVV

SEQ ID NO: 12 - protein, 9 aa, Homo sapiens
FLSNDTVQL

SEQ ID NO: 13 - protein, 137 aa, Mus musculus
NIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 14 - protein, 173 aa, Mus musculus
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 15 - protein, 269 aa, Artificial Sequence
  synthetic
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKGAGGFKTIFGAGTRLFVKANIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 16 - protein, 304 aa, Artificial Sequence
  synthetic
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSPGNTEAFFGQGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 17 - protein, 304 aa, Artificial Sequence
  synthetic
MATSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSPGNTEAFFGQGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 18 - protein, 248 aa, Artificial Sequence
  synthetic
QKEVEQNSGPLSVPEGAIASLNCTYSDRGSQSFFWYRQYSGKSPELIMFIYSNGDKEDGRFTAQLNKASQYVSLLIRDSQPSDSATYLCAVKGAGGFKTIFGAGTRLFVKANIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 19 - protein, 285 aa, Artificial Sequence
  synthetic
DTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSPGNTEAFFGQGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 20 - protein, 137 aa, Artificial Sequence
  synthetic
  MISC_FEATURE (48)..(48): Xaa is Thr or Cys
  MISC_FEATURE (112)..(112): Xaa is Ser, Ala, Val, Leu, Ile, Pro, Phe, Met, or Trp
  MISC_FEATURE (114)..(114): Xaa is Met, Ala, Val, Leu, Ile, Pro, Phe, or Trp
  MISC_FEATURE (115)..(115): Xaa is Gly, Ala, Val, Leu, Ile, Pro, Phe, Met, or Trp
NIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 21 - protein, 173 aa, Artificial Sequence
  synthetic
  MISC_FEATURE (57)..(57): Xaa is Ser or Cys
EDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 22 - protein, 269 aa, Artificial Sequence
  synthetic
  MISC_FEATURE (180)..(180): Xaa is Thr or Cys
  MISC_FEATURE (244)..(244): Xaa is Ser, Ala, Val, Leu, Ile, Pro, Phe, Met, or Trp
  MISC_FEATURE (246)..(246): Xaa is Met, Ala, Val, Leu, Ile, Pro, Phe, or Trp
  MISC_FEATURE (247)..(247): Xaa is Gly, Ala, Val, Leu, Ile, Pro, Phe, Met, or Trp
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKGAGGFKTIFGAGTRLFVKANIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 23 - protein, 304 aa, Artificial Sequence
  synthetic
  MISC_FEATURE (188)..(188): Xaa is Ser or Cys
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSPGNTEAFFGQGTRLTVVEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 24 - protein, 304 aa, Artificial Sequence
  synthetic
  MISC_FEATURE (188)..(188): Xaa is Ser or Cys
MATSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSPGNTEAFFGQGTRLTVVEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 25 - protein, 248 aa, Artificial Sequence
  synthetic
  MISC_FEATURE (159)..(159): Xaa is Thr or Cys
  MISC_FEATURE (223)..(223): Xaa is Ser, Ala, Val, Leu, Ile, Pro, Phe, Met, or Trp
  MISC_FEATURE (225)..(225): Xaa is Met, Ala, Val, Leu, Ile, Pro, Phe, or Trp
  MISC_FEATURE (226)..(226): Xaa is Gly, Ala, Val, Leu, Ile, Pro, Phe, Met, or Trp
QKEVEQNSGPLSVPEGAIASLNCTYSDRGSQSFFWYRQYSGKSPELIMFIYSNGDKEDGRFTAQLNKASQYVSLLIRDSQPSDSATYLCAVKGAGGFKTIFGAGTRLFVKANIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 26 - protein, 285 aa, Artificial Sequence
  synthetic
  MISC_FEATURE (169)..(169): Xaa is Ser or Cys
DTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSPGNTEAFFGQGTRLTVVEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 27 - protein, 27 aa, Artificial Sequence
  synthetic
RAKRSGSGATNFSLLKQAGDVEENPGP

SEQ ID NO: 28 - protein, 600 aa, Artificial Sequence
  synthetic
MATSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSPGNTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKGAGGFKTIFGAGTRLFVKANIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 29 - DNA, 397 bp, Homo sapiens
atgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaaaggtgctggaggcttcaaaactatctttggagcaggaacaagactatttgttaaagcaa

SEQ ID NO: 30 - DNA, 394 bp, Homo sapiens
atgggcaccagcctcctctgctggatggccctgtgtctcctgggggcagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcccgggtaacactgaagctttctttggacaaggcaccagactcacagttgtag

SEQ ID NO: 31 - protein, 9 aa, Homo sapiens
LLLLVLEYL

SEQ ID NO: 32 - protein, 9 aa, Homo sapiens
LLGPWLLLL

SEQ ID NO: 33 - protein, 10 aa, Homo sapiens
HLLGPWLLLL

SEQ ID NO: 34 - protein, 10 aa, Homo sapiens
FLCMSLANPL

SEQ ID NO: 35 - protein, 9 aa, Artificial Sequence
  synthetic
ALSNDTVQL

SEQ ID NO: 36 - protein, 9 aa, Artificial Sequence
  synthetic
FASNDTVQL

SEQ ID NO: 37 - protein, 9 aa, Artificial Sequence
  synthetic
FLANDTVQL

SEQ ID NO: 38 - protein, 9 aa, Artificial Sequence
  synthetic
FLSADTVQL

SEQ ID NO: 39 - protein, 9 aa, Artificial Sequence
  synthetic
FLSNATVQL

SEQ ID NO: 40 - protein, 9 aa, Artificial Sequence
  synthetic
FLSNDAVQL

SEQ ID NO: 41 - protein, 9 aa, Artificial Sequence
  synthetic
FLSNDTAQL

SEQ ID NO: 42 - protein, 9 aa, Artificial Sequence
  synthetic
FLSNDTVAL

SEQ ID NO: 43 - protein, 9 aa, Artificial Sequence
  synthetic
FLSNDTVQA

SEQ ID NO: 44 - protein, 10 aa, Homo sapiens
QDLANDTVQL

SEQ ID NO: 45 - protein, 9 aa, Homo sapiens
LANDTVQLL

SEQ ID NO: 46 - protein, 10 aa, Homo sapiens
MASNDTPEGV

SEQ ID NO: 47 - protein, 9 aa, Homo sapiens
ALSNDTVYV

SEQ ID NO: 48 - protein, 10 aa, Homo sapiens
VALSNDTVYV

SEQ ID NO: 49 - protein, 10 aa, Homo sapiens
TLRNDTVQGV

SEQ ID NO: 50 - protein, 10 aa, Homo sapiens
FIYTLRNDTV

SEQ ID NO: 51 - protein, 10 aa, Homo sapiens
YILENDTVQC

SEQ ID NO: 52 - protein, 9 aa, Homo sapiens
LLSQDTSLT

SEQ ID NO: 53 - protein, 10 aa, Homo sapiens
SQLLSQDTSL

SEQ ID NO: 54 - protein, 10 aa, Homo sapiens
VRLNDTIQLL

SEQ ID NO: 55 - protein, 9 aa, Homo sapiens
RLNDTIQLL

SEQ ID NO: 56 - protein, 10 aa, Homo sapiens
RLNDTIQLLV

SEQ ID NO: 57 - protein, 9 aa, Homo sapiens
PLSADTMWI

SEQ ID NO: 58 - protein, 9 aa, Homo sapiens
PLSSDTMWI

SEQ ID NO: 59 - protein, 9 aa, Homo sapiens
WLTNHTVQL

SEQ ID NO: 60 - protein, 9 aa, Homo sapiens
FLSDDTLFL

SEQ ID NO: 61 - protein, 10 aa, Homo sapiens
LFLSDDTLFL

SEQ ID NO: 62 - protein, 10 aa, Homo sapiens
FLSDDTLFLT

SEQ ID NO: 63 - protein, 10 aa, Homo sapiens
SASEDFTVQL

SEQ ID NO: 64 - protein, 9 aa, Homo sapiens
LLQDTVQQL

SEQ ID NO: 65 - protein, 10 aa, Homo sapiens
RLLQDTVQQL

SEQ ID NO: 66 - protein, 9 aa, Homo sapiens
YITNDTVQT

SEQ ID NO: 67 - protein, 11 aa, Homo sapiens
FLGNFPDTAQL

SEQ ID NO: 68 - protein, 10 aa, Homo sapiens
SLTDDTVQGV

SEQ ID NO: 69 - protein, 9 aa, Homo sapiens
FLSNDAFEI

SEQ ID NO: 70 - protein, 10 aa, Homo sapiens
QFLSNDAFEI

SEQ ID NO: 71 - protein, 10 aa, Homo sapiens
FLSNDAFEIW

SEQ ID NO: 72 - protein, 9 aa, Homo sapiens
FLSNDDYAV

SEQ ID NO: 73 - protein, 10 aa, Homo sapiens
FLSNDDYAVY

SEQ ID NO: 74 - protein, 10 aa, Homo sapiens
DFLSNDDYAV

SEQ ID NO: 75 - protein, 11 aa, Homo sapiens
FLSNDDYAVYV

SEQ ID NO: 76 - protein, 10 aa, Homo sapiens
FLSNDDLLEI

SEQ ID NO: 77 - protein, 10 aa, Homo sapiens
FLSNDELLEI

SEQ ID NO: 78 - protein, 11 aa, Homo sapiens
FLSNDELLEIL

SEQ ID NO: 79 - protein, 9 aa, Homo sapiens
FLFTDTQIV

SEQ ID NO: 80 - protein, 10 aa, Homo sapiens
FLFTDTQIVV

SEQ ID NO: 81 - protein, 10 aa, Homo sapiens
VFLFTDTQIV

SEQ ID NO: 82 - protein, 11 aa, Homo sapiens
VFLFTDTQIVV

SEQ ID NO: 83 - protein, 10 aa, Homo sapiens
LLSSGTVAYL

SEQ ID NO: 84 - protein, 8 aa, Homo sapiens
FLFTDTQI

SEQ ID NO: 85 - protein, 10 aa, Homo sapiens
FLSNDEMLEI

SEQ ID NO: 86 - protein, 11 aa, Homo sapiens
FLSNDEMLEIL

SEQ ID NO: 87 - protein, 8 aa, Homo sapiens
FLITDTQI

SEQ ID NO: 88 - protein, 11 aa, Homo sapiens
YLPNDTVESGI

SEQ ID NO: 89 - protein, 9 aa, Homo sapiens
FLASDTVIY

SEQ ID NO: 90 - protein, 8 aa, Homo sapiens
FLASDTVI

SEQ ID NO: 91 - protein, 10 aa, Homo sapiens
FLSHETVGEV

SEQ ID NO: 92 - protein, 9 aa, Homo sapiens
SLSNDTIAI

SEQ ID NO: 93 - protein, 10 aa, Homo sapiens
VSLSNDTIAI

SEQ ID NO: 94 - protein, 10 aa, Homo sapiens
FLSNDSNINL

SEQ ID NO: 95 - protein, 11 aa, Homo sapiens
WFLSNDSNINL

SEQ ID NO: 96 - protein, 10 aa, Homo sapiens
FLTKDGTVQL

SEQ ID NO: 97 - protein, 11 aa, Homo sapiens
IFLTKDGTVQL

SEQ ID NO: 98 - protein, 10 aa, Homo sapiens
FLMSNETVLL

SEQ ID NO: 99 - protein, 9 aa, Homo sapiens
FLMSNETVL

SEQ ID NO: 100 - protein, 9 aa, Homo sapiens
FLSNDAAYT

SEQ ID NO: 101 - protein, 9 aa, Homo sapiens
LLSQDTSFT

SEQ ID NO: 102 - protein, 9 aa, Homo sapiens
LLQDTVQRL

SEQ ID NO: 103 - protein, 10 aa, Homo sapiens
RLLQDTVQRL

SEQ ID NO: 104 - protein, 9 aa, Homo sapiens
ILHMDTVQL

SEQ ID NO: 105 - protein, 10 aa, Homo sapiens
LQHGADTVQL

SEQ ID NO: 106 - protein, 10 aa, Homo sapiens
GLSPDTVQRL

SEQ ID NO: 107 - protein, 9 aa, Homo sapiens
FLRNDTLLC

SEQ ID NO: 108 - protein, 10 aa, Homo sapiens
FILSNETVNI

SEQ ID NO: 109 - protein, 9 aa, Homo sapiens
MLSSDTSQL

SEQ ID NO: 110 - protein, 10 aa, Homo sapiens
FLGNNTVIDI

SEQ ID NO: 111 - protein, 10 aa, Homo sapiens
SVWSNEIVQL

SEQ ID NO: 112 - protein, 9 aa, Homo sapiens
FLPDDVVQY

SEQ ID NO: 113 - protein, 10 aa, Homo sapiens
FLPDDVVQYL

SEQ ID NO: 114 - protein, 9 aa, Homo sapiens
VLSGDLGQL

SEQ ID NO: 115 - protein, 10 aa, Homo sapiens
RVLSGDLGQL

SEQ ID NO: 116 - protein, 10 aa, Homo sapiens
ALSNDTTESL

SEQ ID NO: 117 - protein, 10 aa, Homo sapiens
LIKVNDTVQI

SEQ ID NO: 118 - protein, 9 aa, Homo sapiens
ALSRSNDTV

SEQ ID NO: 119 - protein, 10 aa, Homo sapiens
FHSSNDTVFI

SEQ ID NO: 120 - protein, 9 aa, Homo sapiens
SLHSSNDTV

SEQ ID NO: 121 - protein, 10 aa, Homo sapiens
VIFNHDTSQL

SEQ ID NO: 122 - protein, 10 aa, Homo sapiens
GLRAIDTTQL

SEQ ID NO: 123 - protein, 10 aa, Homo sapiens
ILSNDKVTQL

SEQ ID NO: 124 - protein, 9 aa, Homo sapiens
FLSRDTVHE

SEQ ID NO: 125 - protein, 10 aa, Homo sapiens
FLSNNNVIRI

SEQ ID NO: 126 - protein, 9 aa, Homo sapiens
QQSANTVQL

SEQ ID NO: 127 - protein, 9 aa, Homo sapiens
YCACDTVQL

SEQ ID NO: 128 - protein, 9 aa, Homo sapiens
SVNSDTVQL

SEQ ID NO: 129 - protein, 9 aa, Homo sapiens
FLSNDLDLY

SEQ ID NO: 130 - protein, 9 aa, Homo sapiens
FFLSNDLDL

SEQ ID NO: 131 - protein, 9 aa, Homo sapiens
FLSNDPCVA

SEQ ID NO: 132 - protein, 10 aa, Homo sapiens
KVNDTVQIDL

SEQ ID NO: 133 - protein, 9 aa, Homo sapiens
QISNDVCQL

SEQ ID NO: 134 - protein, 9 aa, Homo sapiens
MISNDIGQL

SEQ ID NO: 135 - protein, 9 aa, Homo sapiens
GLSNDDFQV

SEQ ID NO: 136 - protein, 9 aa, Homo sapiens
VISNDAPQL

SEQ ID NO: 137 - protein, 9 aa, Homo sapiens
FLGDESMEI

SEQ ID NO: 138 - protein, 9 aa, Homo sapiens
ILTQDSLQM

SEQ ID NO: 139 - protein, 9 aa, Homo sapiens
ILTDQGMDL

SEQ ID NO: 140 - protein, 9 aa, Homo sapiens
LLSDQGFDL

SEQ ID NO: 141 - protein, 9 aa, Homo sapiens
ILAQETLDI

SEQ ID NO: 142 - protein, 9 aa, Homo sapiens
VLSQEGINI

SEQ ID NO: 143 - protein, 9 aa, Homo sapiens
WISQESFDV

SEQ ID NO: 144 - protein, 9 aa, Homo sapiens
FLTQESLQL

SEQ ID NO: 145 - protein, 9 aa, Homo sapiens
FITQDSLQL

SEQ ID NO: 146 - protein, 9 aa, Homo sapiens
LLANDTLNI

SEQ ID NO: 147 - protein, 9 aa, Homo sapiens
VVAENALDV

SEQ ID NO: 148 - protein, 9 aa, Homo sapiens
FLSDNTIEV

SEQ ID NO: 149 - protein, 9 aa, Homo sapiens
FYADESYNL

SEQ ID NO: 150 - protein, 9 aa, Homo sapiens
MLANQGIDV

SEQ ID NO: 151 - protein, 9 aa, Homo sapiens
FLGDQTINL

SEQ ID NO: 152 - protein, 9 aa, Homo sapiens
VISNDSLNV

SEQ ID NO: 153 - protein, 9 aa, Homo sapiens
MLGDEAINL

SEQ ID NO: 154 - protein, 9 aa, Homo sapiens
VFSENSIDV

SEQ ID NO: 155 - protein, 9 aa, Homo sapiens
ILSDQSVNF

SEQ ID NO: 156 - protein, 9 aa, Homo sapiens
VISQQSLEL

SEQ ID NO: 157 - protein, 9 aa, Homo sapiens
ILSNETVNI

SEQ ID NO: 158 - protein, 9 aa, Homo sapiens
LLSEETLNI

SEQ ID NO: 159 - protein, 9 aa, Homo sapiens
FVSQDSLQL

SEQ ID NO: 160 - protein, 9 aa, Homo sapiens
VLSEQTLQV

SEQ ID NO: 161 - protein, 9 aa, Homo sapiens
ILTDEAFEF

SEQ ID NO: 162 - protein, 9 aa, Homo sapiens
ILSNQTVDI

SEQ ID NO: 163 - protein, 9 aa, Homo sapiens
FITDQGIEF

SEQ ID NO: 164 - protein, 9 aa, Homo sapiens
FVTQQAFDI

SEQ ID NO: 165 - protein, 9 aa, Homo sapiens
LVTNQAFDI

SEQ ID NO: 166 - protein, 9 aa, Homo sapiens
LLAEQGYEV

SEQ ID NO: 167 - protein, 9 aa, Homo sapiens
LISDETYNI

SEQ ID NO: 168 - protein, 9 aa, Homo sapiens
YMGQNALQL

SEQ ID NO: 169 - protein, 9 aa, Homo sapiens
LMTQNAVNL

SEQ ID NO: 170 - protein, 9 aa, Homo sapiens
MLAQETLQL